(19) United States
(12) Patent Application Publication
(10) Pub. No.: US 2006/0140970 A1
(43) Pub. Date: Jun. 29, 2006

(54) Proteic binding partners of TCTP and methods of modulating tumor reversion or cell apoptosis

(76) Inventors: Adam Telerman, Paris (FR); Robert Amson, Paris (FR); Christophe Cans, Paris (FR); Vanessa Nancy-Portebois, Pavillons Sous Bois (FR); Brent Passer, Paris (FR)

Correspondence Address: JONES DAY, 51 Louisiana Aveue, N.W, WASHINGTON, DC, 20001-2113, US

(21) Appl. No.: 11/187,891

(22) Filed: Jul. 25, 2005

Related U.S. Application Data
(63) Continuation-in-part of application No. PCT/US04/01894, filed on Jan. 23, 2004.
(60) Provisional application No. 60/441,770, filed on Jan. 23, 2003.
(60) Provisional application No. 60/599,013, filed on Aug. 6, 2004.

(51) Int. Cl. A61K 38/17; A61K 39/00; A61K 38/44; A61K 38/47; A61K 38/18; A61K 38/19

(52) U.S. Cl. 424/185.1; 514/12; 424/94.4; 424/94.61

(57) ABSTRACT

This invention encompasses proteic binding partners of Translationally Controlled Tumor Protein (TCTP), which can modulate tumor reversion or cell apoptosis. Methods of screening for compounds useful for treating, preventing or managing cancer, an inflammatory disorder, or a neurodegenerative disorder and use of these compounds are also encompassed.

This application is a continuation-in-part of PCT/US04/01894, filed Jan. 23, 2004, which claims priority to U.S. Provisional Application No. 60/441,770, filed Jan. 23, 2003, the entirety of which is incorporated herein by reference. This application also claims priority to U.S. Provisional Application No. 60/599,013, filed Aug. 6, 2004, the entirety of which is also incorporated herein by reference.

### 1. FIELD OF THE INVENTION

This invention encompasses methods of modulating tumor reversion or cell apoptosis. Also encompassed are methods of identifying compounds useful for treating, preventing and managing cancer or a neurodegenerative disorder.

### 2. BACKGROUND OF THE INVENTION

Cancer is characterized primarily by an increase in the number of abnormal cells derived from a given normal tissue, invasion of adjacent tissues by these abnormal cells, or lymphatic or blood-borne spread of malignant cells to regional lymph nodes and to distant sites (metastasis). Clinical data and molecular biologic studies indicate that cancer is a multi-step that begins with minor preneoplastic changes, which may under certain conditions progress to neoplasia. The neoplastic region may evolve clonically and develop an increasing capacity for invasion, growth, metastasis, and heterogenicity, especially under conditions in which neoplastic cells escape the host's immune surveillance. Roitt et al., Immunology, 17.1-17.12 (₃rd Ed., Mosby, St. Louis, 1993).

Tumor reversion is a process wherein malignant cells revert to more normal phenotypes. The study of tumor reversion has identified genes that are differentially expressed between tumor cells and their revertants. Tuynder et al., Proc. Natl. Acad. Sci. USA, 99(23): 14976-14981 (2002). One of those is the gene tpt1, which produces the Translationally Controlled Tumor Protein (“TCTP”), which has been found to be overexpressed in tumor cells, but not in the revertants. Id.

The role of TCTP in various other disorders has also been implicated. For example, it has been observed that TCTP is likely to be involved in various inflammatory disorders and neurodegenerative disorders. See, e.g., Bommer et al., Intl. J. Biochem. Cell Biol., 36: 379-385 (2004), which is incorporated herein by reference.

Neurodegenerative disorders are characterized by progressive and irreversible loss of neurons from specific regions of the brain. As a group, these disorders are relatively common and represent a substantial medical and social problem. They are primarily disorders of later life, developing in individuals who are neurologically normal, although childhood-onset forms of each of the disorders are recognized.

Certain genes that are activated or suppressed during the cascade leading to tumor reversion or cell apoptosis have been identified and disclosed in, for example, WO 97/22695 and WO 00/08147, both of which are incorporated herein in their entirety by reference. One of the genes that is suppressed in the cascade, “TSIP2,” was later found to be identical to presenilin 1, whose role had been implicated with Alzheimer's disease. Therefore, compounds that modulate tumor reversion or cell apoptosis present a potential venue in searching for an effective treatment, prevention and management of cancer, an inflammatory disorder, or a neurodegenerative disorder.

### 3. SUMMARY OF THE INVENTION

This invention is based, in part, on the inventors' discovery that certain proteins, i.e., proteic binding partners of TCTP, interact with TCTP, and modulate (i.e., enhance/promote or reduce/inhibit) tumor reversion and/or cell apoptosis. Accordingly, this invention encompasses a method of modulating tumor reversion and/or cell apoptosis comprising contacting a tumor cell with an effective amount of a proteic binding partner of TCTP.

This invention also encompasses a method of identifying a compound that reduces or inhibits the binding between TCTP and a proteic binding partner of TCTP. Compounds so identified can be further screened for their efficacy in promoting or reducing/inhibiting tumor reversion or cell apoptosis.

This invention further encompasses methods of treating, preventing or managing cancer, an inflammatory disorder, or a neurodegenerative disorder using the compounds identified using screening methods of this invention. In some embodiments, proteic binding partners of TCTP can be directly used for the treatment, prevention or management. Some proteic binding partners of TCTP, which promotes tumor reversion or cell apoptosis, can be used for the treatment, prevention or management of cancer, while other proteic binding partners of TCTP, which reduces or inhibits tumor reversion or cell apoptosis, can be used for the treatment, prevention or management of a neurodegenerative disorder. In other embodiments, other compounds, selected using methods based on the interaction between TCTP and its proteic binding partners, are used for the treatment, prevention or management of cancer or a neurodegenerative disorder.

This invention also encompasses diagnostic procedures, by which the presence or cause of cancer, an inflammatory disorder, or a neurodegenerative disorder can be determined. These procedures comprise the determination of whether the binding between TCTP and its binding partner is altered. Where the alteration of the binding is observed, the presence of the disorder can be confirmed by, and/or the cause of the disorder can be attributed to, such alteration of binding.

Specific proteic binding partners used in methods of this invention include, but are not limited to:

| PROTEIN | Accession Number |
|---|---|
| Human elongation factor-1 Delta | NM_032378, NM_001960 |
| stathmin | X53305 |
| S100P calcium binding protein | BC006819 |
| casein kinase II alpha subunit | AY112721 |
| beta 2 integrin | BC005861 |
| beta actin | BC016045 |
| prothymosin beta 4 | BC001631 |
| SWI/SNF related, matrix associated, actin | NM_139045 |
| dependent regulator of chromatin | |
| (subfamily a, member 2) | |
| ATP synthase, H+ transporting, | BC022865 |
| mitochondrial F1 complex, O subunit | |
| NADH dehydrogenase (ubiquinone), 1 beta | BC009796 |
| subcomplex 5 | |
| pyruvate dehydrogenase (lipoamide) alpha 1 | BC002406 |
| YME-1 like 1 | BC024032 |
| vascular protein sorting 35 (VPS 35) | NM_018206 |
| ADP-ribosylation factor 1 (ARF 1) | XM_047545 |
| guanine nucleotide binding protein (G | BC021993 |
| protein), beta polypeptide 2-like 1 | |
| coatomer protein complex subunit alpha | U24105 |
| (COPA) | |
| DEAE/H(Asp-Glu-Ala-Asp/His) box | BC016027 |
| polypeptide 5 (RNA helicase) | |
| PAI-1 mRNA binding protein (PAI-RBP1) | NM_015640 |
| Isoleucine t-RNA synthetase | BC008318 |
| cMyb protooncogene | AF104863 |
| Activated RNA polymerase II transcription | BC022339 |
| cofactor 4 | |
| Transcriptional regulating protein 132 | AF297872 |
| (TreP-132) | |
| Eukaryotic translation initiation factor 3, | BC000386 |
| gamma subunit | |
| Topoisomerase II alpha | J04088 |
| Growth factor independence I | BC032751 |
| C-terminal end of the | BC018641 |
| cds-elongation factor I alpha | |
| tRNA nucleotidyl transferase, CCA-adding | AF151805 |
| 1 (TRNT1) | |
| Splicing factor, arginine/serine rich 2 | M90104 |
| Hypoxanthine phosphoribosyltrasferase 1 | BC000578 |
| Dyskerin | BC010015 |
| Human stress-induced phosphoprotein 1 | BC002967 |
| Hsp 70 like protein | AF112210 |
| SH3 domain kinase binding protein 1 | BC015806 |
| (SH3KBP1) | |
| Protein kinase C inhibitor 1 | HSU27143 |
| Pyruvate kinase | BC007952 |
| Glutamate ammonia ligase | BC011852 |
| (glutamine synthase) | |
| Galactosyltransferase I | BC007317 |
| DCAL-1 | NM_172004 |
| Down syndrome critical region 2 | AJ006291 |
| NS-associated protein 1 like protein | AF155568 |
| MHC HLA class I cell surface antigen | M32319 |
| TCTP like protein | BC022436 |
| DHHC1 protein | AF441791 |
| Diazepam binding inhibitor | M14200 |
| HIRA interacting protein 5 | AJ132584 |
| TCAA3 (transforming acidic coiled-coil | AF093543 |
| containing protein 3) | |
| Rad 50 interacting protein | AF317622 |
| Structure specific recognition protein | BC005116 |
| Macrophage galactose-type C-type lectin 2 | XM_090246 |
| like protein | |

In certain embodiments, preferred proteic binding partner of TCTP is OP18 (stathmin) or eEF1A.

### 4. BRIEF DESCRIPTION OF FIGURES

FIG. 1 illustrates the inhibition of GTP-GDP exchange activity of Arf1-GAP by TCTP.

FIG. 2 illustrates the localization of TCTP during mitosis.

FIG. 3A illustrates the mitotic defects caused by overexpression of TCTP in HeLa cells.

FIG. 3B illustrates the delay in metaphase/anaphase transition caused by TCTP overexpression.

FIG. 3C illustrates unsynchronized telophase exit caused by TCTP overexpression.

FIG. 3D illustrates increased cell death rate in interphase caused by TCTP overexpression.

FIG. 4 illustrates the effects of TCTP overexpression on mitotic spindle.

FIG. 5A illustrates the downexpression of TCTP using siRNA.

FIG. 5B illustrates percentage of dividing cells with TCTP downexpression in each mitosis phases.

FIG. 5C illustrates the division of the whole population of cells with TCTP downexpression.

FIG. 5D illustrates the induction of metaphase/anaphase delay by TCTP downexpression.

FIG. 6 illustrates the effects of TCTP on microtubules dynamics in vitro.

FIG. 7A illustrates the result of yeast two hybrid hunt using TCTP as bait, and the identification of OP18 as a binding partner.

FIG. 7B illustrates the interaction of TCTP and OP18 (stathmin) in vivo, using co-precipitation in U937 cells.

FIG. 8 illustrates the mapping of the OP18 region that interacts with TCTP.

FIG. 9 illustrates the co-localization of TCTP and OP18 during mitosis.

FIG. 10 illustrates the modulation of OP18 phosphorylation by TCTP.

FIG. 11A illustrates the validation of the assay for the polymerization of tubulin, and the effects of TCTP on the polymerization.

FIG. 12B illustrates the effects of TCTP overexpression on the polymerization of microtubules and OP 18 phosphorylation in nocodazole treated cells.

FIG. 12A illustrates interaction between LexA-TCTP and B42-eEF1BA or B42-eEF1Bβ.

FIG. 12B illustrates in vitro interaction between ³⁵S-labeled eEF1Bβ and GST-TCTP.

FIG. 12C illustrates in vitro interaction between purified eEF1A and GST-TCTP.

FIG. 13A illustrates an immunoblot analysis using antibodies against eEF1Bβ.

FIG. 13B illustrates co-immunoprecipitation of TCTP and eEF1Bβ using anti-chicken TCTP antibodies.

FIG. 13C illustrates an immunoblot analysis using antibodies against eEF1A.

FIG. 13D illustrates an association between eEF1A and eEF1Bβ.

FIG. 14A illustrates an indirect immunofluorescence study showing endogenous partial co-localization of TCTP and eEF1Bβ.

FIG. 14B illustrates an indirect immunofluorescence study showing endogenous partial co-localization of TCTP and eEF1A.

FIG. 15A illustrates dissociation kinetics of GDP in presence of increasing amounts of TCTP, when eEF1A is preloaded with GDP.

FIG. 15B illustrates the effect of TCTP on the GDP-GTP exchange.

FIG. 15C illustrates the kinetics of GDP exchange inhibition by TCTP.

FIG. 16A illustrates the kinetics of eEF1Bαγ mediated GDP exchange in the presence of TCTP.

FIG. 16B illustrates kinetics of eEF1Bβ mediated GDP exchange in the presence of TCTP.

FIG. 17A illustrates an immunoblot analysis of 293T cell lysates using a rabbit anti-eEF1Bβ serum.

FIG. 17B illustrates an immunoblot analysis of 293T cell lysates using a chicken anti-TCTP antibody.

FIG. 18 illustrates endogenous partial co-localization of TCTP and eEF1Bβ with Phospho-Disulfide-Isomerase.

### 5. DETAILED DESCRIPTION OF THE INVENTION

TCTP is a ubiquitously expressed protein that is evolutionarily conserved. Its role in tumor reversion and/or cell apoptosis has been implicated. Tuynder et al., Proc. Natl. Acad. Sci. USA, 99(23): 14976-81 (2002). Furthermore, TCTP has been reported to be associated with various disorders, such as, but not limited to, cancer, an inflammatory disorder, and a neurodegenerative disorder. This invention is based, in part, on the inventors' discovery that certain proteins, i.e., proteic binding partners of TCTP, can interact with TCTP, and thus may be used to modulate TCTP's effect on tumor reversion and/or cell apoptosis and the disorders associated with TCTP.

A first embodiment of this invention encompasses methods of modulating tumor reversion and/or cell apoptosis comprising contacting a tumor cell with an effective amount of a proteic binding partner of TCTP.

As used herein, and unless otherwise specified, the terms “modulate,” “modulating,” and “modulation,” when used in connection with tumor reversion or cell apoptosis, mean that the extent or intensity of such tumor reversion or cell apoptosis is altered or adjusted. The terms encompass increase, promotion, decrease, reduction and inhibition of tumor reversion or cell apoptosis.

As used herein, or unless otherwise specified, the terms “promote,” “promotion,” and “increase,” when used in connection with tumor reversion or cell apoptosis, mean that tumor reversion or cell apoptosis in a cell, treated using methods of this invention, is higher than the same type of cell without the treatment. In some embodiments, tumor reversion or cell apoptosis in a cell treated using methods of this invention is higher than that in the same type of cell without the treatment by about 10 percent, 30 percent, 50 percent, 70 percent, 100 percent, 200 percent or more, as determined using a well-known analytical method such as, but not limited to, SDS-PAGE, PARP cleavage, spectrophotometry, autoradiography, or chromatographic techniques.

As used herein, and unless otherwise specified, the terms “decrease,” “reduce,” and “reduction,” when used in connection with tumor reversion or cell apoptosis, means that tumor reversion or cell apoptosis in a cell, treated using methods of this invention, is lower than the same type of cell without the treatment. In some embodiments, tumor reversion or cell apoptosis in a cell treated using methods of this invention is lower than that in the same type of cell without the treatment by about 10 percent, 30 percent, 50 percent, 70 percent or 90 percent or more, as determined using a well-known analytical method such as, but not limited to, SDS-PAGE, PARP cleavage, spectrophotometry, autoradiography, or chromatographic techniques.

In this regard, the terms “inhibit” and “inhibition” mean that tumor reversion or cell apoptosis in a cell treated using methods of this invention is substantially abolished. In some embodiments, tumor reversion or cell apoptosis is inhibited where no or little such tumor reversion or cell apoptosis can be detected using a well-known analytical method such as, but not limited to, SDS-PAGE, PARP cleavage, spectrophotometry, autoradiography, or chromatographic techniques.

As used herein, and unless otherwise specified, the term “proteic binding partner of TCTP” means a protein, or portions thereof, that can bind to or interact with TCTP. Any known methods of identification of a binding partner may be used, e.g., co-precipitation and fluorescence resonance energy transfer. A specific method is yeast two-hybrid system, derived from the system described by Finley and Brent (Interaction trap cloning with yeast, in DNA Cloning, Expression Systems: A Practical Approach, pp. 169-203, Oxford Universal Press, Oxford (1995)), which is incorporated herein by reference. Examples of proteic binding partners of TCTP include, but are not limited to, human elongation factor-1 Delta and those listed in Table 1.

5.1 Methods of Screening

In one embodiment, this invention encompasses methods of identifying compounds that reduce or inhibit binding between TCTP and a proteic binding partner of TCTP comprising: (a) contacting a compound with TCTP and the proteic binding partner of TCTP; and (b) determining whether the binding between TCTP and the proteic binding partner of TCTP is decreased.

As used herein, and unless otherwise indicated, the terms “reduce” and “decrease,” when used in connection with binding, mean that the binding between two molecules are impeded, slowed or prevented in the presence of a test compound as compared to the binding occurring in the absence of such a test compound. Binding between two molecules may be determined by any methods known in the art, including, but not limited to, fluorescence, spectroscopy, chromatography, centrifugation and electrophoresis. In some embodiments, for a test compound to be identified positive, binding between TCTP and a proteic binding partner of TCTP is reduced by about 10 percent, 20 percent, 40 percent, 60 percent, 80 percent or 90 percent or more in the presence of the test compound as compared to the binding of the same molecules in the absence of the test compound.

In another embodiment, this invention encompasses methods of identifying compounds that are useful in treating, preventing or managing cancer. One aspect of this embodiment encompasses methods of identifying compounds that promote tumor reversion or cell apoptosis comprising: (a) contacting a proteic binding partner of TCTP with a tumor cell; and (b) determining whether tumor reversion or cell apoptosis is increased as compared to a tumor cell that has not been contacted with the proteic binding partner of TCTP. The proteic binding partner identified positive using the methods can be used in treating, preventing or managing cancer.

In another aspect of this embodiment, this invention also encompasses methods of identifying compounds that promote tumor reversion or cell apoptosis comprising: (a) contacting a compound with TCTP and a proteic binding partner of TCTP; (b) determining whether binding between TCTP and the proteic binding partner of TCTP is altered. The methods may further comprise: contacting the compound identified in steps (a) and (b) with a tumor cell; and determining whether tumor reversion or cell apoptosis is increased as compared to a tumor cell that has not been contacted with the compound. Compounds tested positive using this method is potentially useful for the treatment, prevention and/or management of cancer.

In another embodiment, this invention encompasses methods of identifying compounds that are useful in treating, preventing or managing an inflammatory disorder. In one aspect of this embodiment, this invention encompasses methods of identifying compounds useful for treating, preventing and/or managing an inflammatory disorder comprising: (a) contacting a compound with TCTP and a proteic binding partner of TCTP; (b) determining whether binding between TCTP and the proteic binding partner of TCTP is altered. The methods may comprise further steps of determining whether the compound identified in steps (a) and (b) has anti-inflammatory activity.

Any procedures for determination of anti-inflammatory activity known in the art may be used in connection with the methods of the invention. Examples of such procedures include, but are not limited to, chemical (e.g., carrageenin) induced edema test, antigranuloma test, antiexudation test, and antiadjuvant-arthritis test, all of which are described in U.S. Pat. No. 4,380,536, incorporated herein by reference.

In another embodiment, this invention also encompasses methods of identifying compounds potentially useful in the treatment, prevention and/or management of a neurodegenerative disorder such as Alzheimer's disease. One aspect of this embodiment encompasses methods of identifying compounds useful for the treatment, prevention, and/or management of a neurodegenerative disorder comprising: (a) contacting a proteic binding partner of TCTP with a cell; and (b) determining whether cell apoptosis is reduced as compared to a cell that has not been contacted with the proteic binding partner of TCTP.

In another aspect of this embodiment, this invention also encompasses methods of identifying compounds useful for the treatment, prevention, and/or management of a neurodegenerative disorder comprising: (a) contacting a compound with TCTP and a proteic binding partner of TCTP; (b) determining whether binding between TCTP and the proteic binding partner of TCTP is altered. The methods may further comprise: (c) contacting the compound identified from steps (a) and (b) with a cell; and (d) determining whether cell apoptosis is reduced as compared to a cell that has not been contacted with the compound. The compound identified using this method may be useful in the treatment, prevention, and/or management of a neurodegenerative disorder.

This invention also encompasses methods of determining the cause of cancer, an inflammatory disorder, or a neurodegenerative disorder. The methods comprise: (a) obtaining a first cell from a patient suffering from said disorder; (b) determining the binding between TCTP and its proteic binding partner in the first cell; (c) obtaining a second cell from a normal subject; (d) determining the binding between TCTP and its proteic binding partner in the second cell; and (e) comparing the bindings obtained from the first cell and the second cell and determining whether the binding in the first cell is altered. Where the alteration of the binding is observed, the cause of the disorder can be attributed to, such alteration of binding.

This invention also encompasses a method of diagnosing cancer or predicting the propensity for occurrence of cancer in a subject comprising: (a) obtaining a first cell from a potential patient; (b) determining the binding between TCTP and a proteic binding partner in the first cell; (c) obtaining a second cell from a normal subject; (d) determining the binding between TCTP and the proteic binding partner in the second cell; and (e) comparing the bindings obtained from the first cell and the second cell and determining whether the binding in the first cell is altered. Where the binding is altered, the potential patient is diagnosed for cancer or can be predicted to have a higher than normal propensity for the occurrence of cancer.

In all of the embodiment, the binding between TCTP and its proteic binding partner may be determined by any methods known in the art, including, but not limited to, fluorescence, spectroscopy, chromatography, centrifugation and electrophoresis.

Whether tumor reversion or cell apoptosis increases or decreases in the tumor cell contacted with the test compound can be determined by any methods known in the art, as well as those described herein. The determination can be done in vivo or in vitro. For example, where the tests are carried out in vitro, models such as K256/KS cells described in Telerman et al., Proc. Natl. Acad. Sci. USA, 90: 8702-6 (1993), M1-LTR cells described in Amson et al., Proc. Natl. Acad. Sci. USA, 93: 3953-7 (1996), and U937/US3-US4 cells described in Nemani et al., Proc. Natl. Acad. Sci. USA, 93: 9039-42 (1996), all of which are incorporated herein by reference, may be used. Cell apoptosis may also be assessed using non-tumor cells. In the case of in vivo tests, the test compound can be injected into an animal model, such as immunodepressed mice, and effects of the injected test compound may be investigated. Specific conditions and thresholds for identification are well within the ordinary skill in the art.

In the case of cell apoptosis, a specific method that can be used is Poly ADP-Ribose Polymerase (PARP) cleavage test. In short, cells are treated with a test compound, and proteins are isolated. An anti-PARP can be used to visualize the location of PARP, which, in turn, provides an indication of whether PARP cleavage has occurred or not. Cleavage of PARP is an indication of the induction of cell apoptosis.

In a specific embodiment, the proteic binding partners that can be used in connection with the methods of this invention are those listed in Table 1, below. In another specific embodiment, the proteic binding partners that can be used in connection with the methods of this invention is OP18 (stathmin) or eukaryotic elongation factor 1A (eEF1A).

5.2 Methods of Treatment, Prevention and Management

In one embodiment, this invention encompasses methods of treating, preventing and/or managing cancer comprising administering to a patient in need of such treatment, prevention or management a therapeutically or prophylactically effective amount of a compound identified using certain methods of this invention.

As used herein, and unless otherwise specified, the terms “treat,” “treating” and “treatment” contemplate an action that occurs while a patient is suffering from the specified disease or disorder, which reduces the severity of the disease or disorder, or retards or slows the progression of the disease or disorder. The terms refer to the eradication or amelioration of a disease or condition, or of one or more symptoms associated with the disease or condition. In certain embodiments, the terms refer to minimizing the spread or worsening of the disease or condition resulting from the administration of one or more prophylactic or therapeutic agents to a subject with such a disease or condition.

As used herein, and unless otherwise specified, the terms “prevent,” “preventing” and “prevention” contemplate an action that occurs before a patient begins to suffer from the specified disease or disorder, which inhibits or reduces the severity of the disease or disorder. The terms also refer to the prevention of the onset, recurrence or spread of a disease or condition, or of a symptom thereof.

As used herein, and unless otherwise specified, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

As used herein and unless otherwise indicated, the term “managing” encompasses preventing the recurrence of cancer in a patient who had suffered from cancer, lengthening the time a patient who had suffered from cancer remains in remission, preventing the occurrence of cancer in patients at risk of suffering from cancer (e.g., patients who had been exposed to high amounts of radiation or carcinogenic materials, such as asbestos; patients infected with viruses associated with the occurrence of cancer, such as, but not limited to, HIV and Kaposi's sarcoma-associated herpes virus; and patients with genetic predispositions to cancer, such as those suffering from Downs syndrome), and preventing the occurrence of malignant cancer in patients suffering from pre-malignant or non-malignant cancers.

As used herein, and unless otherwise specified, a “therapeutically effective amount” of a compound is an amount sufficient to provide a therapeutic benefit in the treatment or management of a disease or condition, or to delay or minimize one or more symptoms associated with the disease or condition. A therapeutically effective amount of a compound means an amount of therapeutic agent, alone or in combination with other therapies, which provides a therapeutic benefit in the treatment or management of the disease or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease or condition, or prevent its recurrence. A prophylactically effective amount of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

As used herein, the term “cancer” includes, but is not limited to, solid tumors and blood born tumors. The term “cancer” refers to disease of skin tissues, organs, blood, and vessels, including, but not limited to, cancers of the bladder, bone or blood, brain, breast, cervix, chest, colon, endrometrium, esophagus, eye, head, kidney, liver, lymph nodes, lung, mouth, neck, ovaries, pancreas, prostate, rectum, stomach, testis, throat, and uterus. Specific cancers include, but are not limited to, advanced malignancy, amyloidosis, neuroblastoma, meningioma, hemangiopericytoma, multiple brain metastases, glioblastoma multiforms, glioblastoma, brain stem glioma, poor prognosis malignant brain tumor, malignant glioma, recurrent malignant giolma, anaplastic astrocytoma, anaplastic oligodendroglioma, neuroendocrine tumor, rectal adenocarcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, localized or metastatic melanoma (of any kind, including, but not limited to, ocular), peritoneal carcinoma, papillary serous carcinoma, gynecologic sarcoma, soft tissue sarcoma, scelroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waldenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma. In a specific embodiment, the cancer is metastatic. In another embodiment, the cancer is refractory or resistance to chemotherapy or radiation.

In some embodiments of this invention, compounds of this invention can be administered, sequentially or simultaneously, with another anticancer agent. The administration may be via same route or different routes. Examples of anti-cancer agents include, but are not limited to: acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., Gleevec®), imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim;Erbitux, human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (Genasense®); O⁶-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras famesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

The magnitude of a prophylactic or therapeutic dose of each active ingredient in the treatment, prevention and/or management of cancer will typically vary with the specific active ingredients, the severity and type of cancer, and the route of administration. The dose, and perhaps the dose frequency, may also vary according to age, body weight, response, and the past medical history of the patient. Suitable dosing regimens can be readily selected by those skilled in the art with due consideration of such factors by following, for example, dosages reported in the literature and recommended in the Physician's Desk Reference® (54^{th} ed., 2000).

In one embodiment, the patient is an animal. In another embodiment, the patient is a mammal, specifically, a human.

In another embodiment, this invention encompasses methods of treating, preventing and managing an inflammatory disorder comprising administering to a patient in need of such treatment, prevention and/or management a therapeutically or prophylactically effective amount of a compound identified certain methods of this invention.

Examples of inflammatory disorders include, but are not limited to: inflammation, including rheumatoid arthritis; pain, including neuropathic pain; gout; endotoxic shock; bronchoconstriction; coronary artery disease; thrombosis; an allergic disorder such as rhinitis, allergic dermatosis, urticaria, angioedema, and atopic dermatitis; motion sickness; vertigo; asthma; a vascular disease, such as atherosclerosis, Raynaud's phenomenon, spasm of arteries, and hypertension. In one embodiment, the patient is a mammal, in particular, a human.

In some embodiments, compound of this invention may be administered, sequentially or simultaneously, with another agent useful in treating a neurodegenerative disorder. The administration may be via same route or different routes. Examples of other agents include, but are not limited to: other antiinflammatory agents such as, but not limited to, ibuprofen, naproxen, fenoprofen, ketoprofen, flurbiprofen, piroxicam, diclofenac, etodolac, nabumetone, and colchicine; glucocorticoids; immunosuppressive agents such as, but not limited to, azathiprine and methotrexate; analgesic or antipyretic agents such as, but not limited to, salicylic acid, aspirin, diflunisal, phenylbutazone, oxyphenylbutazone, phenazone, aminopyrine, azapropazone, acetaminophen, indomethacin, sulindac, mefenamic acid, and tolmetin; H₁ antagonists such as, but not limited to, diphenhydramine, chlorpheniramine, pyrilamine, chlorcyclizine, promethazine, terfenadine, astemizole, and meclizine; and 5-HT antagonists such as, but not limited to, ketanserin, methysergide, and cyproheptadine.

The magnitude of a prophylactic or therapeutic dose of each active ingredient in the treatment, prevention and/or management of an inflammatory disorder will typically vary with the specific active ingredients, the severity and type of disorder, and the route of administration. The dose, and perhaps the dose frequency, may also vary according to age, body weight, response, and the past medical history of the patient. Suitable dosing regimens can be readily selected by those skilled in the art with due consideration of such factors by following, for example, dosages reported in the literature and recommended in the Physician's Desk Reference® (54^{th} ed., 2000).

In another embodiment, this invention encompasses methods of treating, preventing and managing a neurodegenerative disorder comprising administering to a patient in need of such treatment, prevention and/or management a therapeutically or prophylactically effective amount of a compound identified certain methods of this invention.

Examples of neurodegenerative disorders that can be treated using methods of this invention include, but are not limited to, amylotrophic lateral sclerosis, Alzheimer's disease, Huntington's disease and Parkinson's disease. In one embodiment, the neurodegenerative disorder that can be treated, prevented and/or managed using methods of this invention is Alzheimer's disease. In one embodiment, the patient is a mammal, in particular, a human.

In some embodiments, compound of this invention may be administered, sequentially or simultaneously, with another agent useful in treating a neurodegenerative disorder. The administration may be via same route or different routes. Examples of other agents include, but are not limited to: acridine derivatives such as tacrine; amantadine; dopamine depleting agents such as tetrabenazine and reserpine; dopamine receptor antagonists such as bromocriptine and pergolide; metabolic precursors of dopamine such as levodopa; inhibitors of acetylcholinesterase (AChE) such as physostigmine; inhibitors of L-amino acid decarboxylase such as benserazide and carbidopa; muscarinic receptor antagonists such as benzotropine mesylate, diphenhydramine hydorchloride and trihexyphenidyl; precursors of acetylcholine synthesis such as choline chloride and phosphatidyl choline (lecithin); and selegiline.

The magnitude of a prophylactic or therapeutic dose of each active ingredient in the treatment, prevention and/or management of a neurodegenerative disorder will typically vary with the specific active ingredients, the severity and type of disorder, and the route of administration. The dose, and perhaps the dose frequency, may also vary according to age, body weight, response, and the past medical history of the patient. Suitable dosing regimens can be readily selected by those skilled in the art with due consideration of such factors by following, for example, dosages reported in the literature and recommended in the Physician's Desk Reference® (54^{th} ed., 2000).

5.3 Pharmaceutical Compositions

In some embodiments, this invention encompasses pharmaceutical compositions comprising one or more proteic binding partners of TCTP, or genes thereof. In other embodiments, this invention also encompasses pharmaceutical compositions comprising one or more of compounds identified using methods of this invention. Such pharmaceutical compositions are described below.

Certain pharmaceutical compositions are single unit dosage forms suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The formulation should suit the mode of administration. For example, oral administration requires enteric coatings to protect the compounds of this invention from degradation within the gastrointestinal tract. In another example, the compounds of this invention may be administered in a liposomal formulation to shield the compounds from degradative enzymes, facilitate transport in circulatory system, and effect delivery across cell membranes to intracellular sites.

The composition, shape, and type of dosage forms of the invention will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

5.3.1 Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the active ingredients in an intimate admixture with at least one excipient according to conventional pharmaceutical compounding techniques. Excipients can take a wide variety of forms depending on the form of preparation desired for administration.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

Disintegrants or lubricants can be used in pharmaceutical compositions and dosage forms of the invention.

5.3.2 Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients (i.e., the compounds of this invention) disclosed herein can also be incorporated into the parenteral dosage forms of the invention.

5.3.3 Transdermal, Topical and Mucosal Dosage Forms

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington 's Pharmaceutical Sciences, 16th and 18th eds., Mack Publishing, Easton Pa, (1980 & 1990); and Introduction to Pharmaceutical Dosage Forms, 4th ed., Lea & Febiger, Philadelphia (1985). Transdernal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts, and depend on the particular tissue to which a given pharmaceutical composition or dosage form will be applied.

Depending on the specific tissue to be treated, additional components may be used prior to, in conjunction with, or subsequent to treatment with active ingredients of the invention. For example, penetration enhancers can be used to assist in delivering the active ingredients to the tissue.

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

5.3.4 Compositions with Enhanced Stability

The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, this invention encompasses pharmaceutical compositions and dosage forms that contain little, if any, lactose other mono- or di-saccharides. As used herein, the term “lactose-free” means that the amount of lactose present, if any, is insufficient to substantially increase the degradation rate of an active ingredient.

Lactose-free compositions of the invention can comprise excipients that are well known in the art and are listed, for example, in the U.S. Pharmacopeia (USP) 25-NF20 (2002). In general, lactose-free compositions comprise active ingredients, a binder/filler, and a lubricant in pharmaceutically compatible and pharmaceutically acceptable amounts. Preferred lactose-free dosage forms comprise active ingredients, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients.

5.3.5 Delayed Release Dosage Forms

Active ingredients of the invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the compounds of this invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

5.3.6 Delivery of Nucleic Acids

Active ingredients in pharmaceutical compositions of this invention may be in the form of nucleic acids. Delivery of these molecules into a patient can either be direct, i.e., the patient is directly exposed to the compounds of this invention or compound-carrying vector, or indirect, i.e., cells are first transformed with the compounds of this invention in vitro, then transplanted into the patient for cell replacement therapy. These two approaches are known as in vivo and ex vivo therapy, respectively.

In the case of in vivo therapy, compounds of this invention are directly administered in vivo, where they are expressed to produce the encoded product. This can be accomplished by any of numerous methods known in the art, e.g., by constructing them as part of an appropriate nucleic acid expression vector and administering them so that they become intracellular, by infection using a defective or attenuated retroviral or other viral vector (see, e.g., U.S. Pat. No. 4,980,286), by direct injection of naked DNA, by use of microparticle bombardment (e.g., a gene gun; Biolistic®, DuPont), by coating with lipids or cell-surface receptors or transfecting agents, encapsulation in liposomes, microparticles, or microcapsules, by administering them in linkage to a peptide which is known to enter the cell or nucleus, or by administering them in linkage to a ligand subject to receptor-mediated endocytosis (Wu and Wu, J. Biol. Chem., 262: 4429-32 (1987)), which can be used to target cell types specifically expressing the receptors. Further, compounds of this invention can be targeted in vivo for cell specific uptake and expression, by targeting a specific receptor, as disclosed in, for example, WO 92/06180, WO 92/22635, WO 92/20316, WO 93/14188 and WO 93/20221, all of which are incorporated herein by reference.

Ex vivo therapy involves transferring the compounds of this invention to cells in tissue culture by methods such as electroporation, lipofection, calcium phosphate mediated transfection and viral infection. Usually, the method of transfer includes the transfer of a selectable marker to the cells. The cells are then placed under selection to isolate those cells that have taken up and are expressing the transferred compounds. These cells are then delivered to a patient.

Compounds of this invention are introduced into a cell prior to administration in vivo of the resulting recombinant cell. Such introduction can be carried out by any methods known in the art, including, but not limited to, transfection, electroporation, microinjection, infection with a viral vector containing the nucleic acid sequences, cell fusion, chromosome-mediated gene transfer, microcell-mediated gene transfer and spheroplast fusion. Numerous techniques are known in the art for the introduction of foreign compounds into cells. Examples of such techniques are disclosed in: Loeffler et al., Meth. Enzymol., 217: 599-618 (1993); Cohen et al., Meth. Enzymol., 217: 618-644 (1993); and Cline, Pharmc. Ther., 29: 69-92 (1985), all of which are incorporated herein by reference. These techniques should provide for the stable transfer of the compounds of this invention to the cell, so that they are expressible by the cell and preferably heritable and expressible by its cell progeny.

The resulting recombinant cells can be delivered to a patient by various methods known in the art. Examples of the delivery methods include, but are not limited to, subcutaneous injection, skin graft and intravenous injection.

5.3.7 Kits

In some cases, active ingredients of the invention are preferably not administered to a patient at the same time or by the same route of administration. This invention therefore encompasses kits which, when used by the medical practitioner, can simplify the administration of appropriate amounts of active ingredients to a patient.

A typical kit of the invention comprises a single unit dosage form of the compounds of this invention, or a pharmaceutically acceptable salt, prodrug, solvate, hydrate, clathrate or stereoisomer thereof, and a single unit dosage form of another agent that may be used in combination with the compounds of this invention. Kits of the invention can further comprise devices that are used to administer the active ingredients. Examples of such devices include, but are not limited to, syringes, drip bags, patches, and inhalers.

Kits of the invention can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

The invention is further defined by reference to the following non-limiting examples. It will be apparent to those skilled in the art that many modifications, both to materials and methods, can be practiced without departing from the spirit and scope of this invention.

### 6. EXAMPLES

6.1 Identification of Proteic Binding Partners of TCTP

Two separate yeast two hybrid hunts were performed with TCTP. One involved using the full-length TCTP as bait, and the second involved using the first 80 amino acids of TCTP as bait. A cDNA library derived from U937 cell line was used to identify the proteic binding partners of TCTP.

When the full-length TCTP was used, human elongation factor-1 delta (Accession No. NM_{—}032378; NM_{—}001960) was identified as the binding partner of TCTP. The results from the two hybrid hunt using the first 80 amino acids of TCTP are shown below in Table 1.

TABLE 1
Proteic Binding Partners of TCTP

| | Accession | Size in | Size in |
|---|---|---|---|
| PROTEIN | Number | Amino Acids | Base Pairs |
| stathmin | X53305 | 150 | 450 |
| S100P calcium binding protein | BC006819 | 96 | 288 |
| casein kinase II alpha subunit | AY112721 | 392 | 1176 |
| beta 2 integrin | BC005861 | 770 | 2310 |
| beta actin | BC016045 | 376 | 1128 |
| prothymosin beta 4 | BC001631 | 193 | 579 |
| SWI/SNF related, matrix | NM_139045 | 1569 | 4707 |
| associated, actin dependent | | | |
| regulator of chromatin | | | |
| (subfamily a, member 2) | | | |
| ATP synthase, H+ | BC022865 | 214 | 642 |
| transporting, mitochondrial F1 | | | |
| complex, O subunit | | | |
| NADH dehydrogenase | BC009796 | 190 | 570 |
| (ubiquinone), 1 beta | | | |
| subcomplex 5 | | | |
| pyruvate dehydrogenase | BC002406 | 391 | 1173 |
| (lipoamide) alpha 1 | | | |
| YME-1 like 1 | BC024032 | 717 | 2151 |
| vascular protein sorting 35 | NM_018206 | 797 | 2391 |
| (VPS 35) | | | |
| ADP-ribosylation factor 1 | XM_047545 | 182 | 546 |
| (ARF 1) | | | |
| guanine nucleotide binding | BC021993 | 318 | 954 |
| protein (G protein), beta | | | |
| polypeptide 2-like 1 | | | |
| coatomer protein complex | U24105 | 1125 | 3675 |
| subunit alpha (COPA) | | | |
| DEAE/H(Asp-Glu-Ala- | BC016027 | 615 | 1845 |
| Asp/His) box polypeptide 5 | | | |
| (RNA helicase) | | | |
| PAI-1 mRNA binding protein | NM_015640 | 388 | 1164 |
| (PAI-RBP1) | | | |
| Isoleucine t-RNA synthetase | BC008318 | 365 | 1095 |
| cMyb protooncogene | AF104863 | 641 | 1923 |
| Activated RNA polymerase II | BC022339 | 128 | 384 |
| transcription cofactor 4 | | | |
| Transcriptional regulating | AF297872 | 1201 | 3603 |
| protein 132 | | | |
| (TreP-132) | | | |
| Eukaryotic translation | BC000386 | 353 | 1058 |
| initiation factor 3, gamma | | | |
| subunit | | | |
| Topoisomerase II alpha | J04088 | 1532 | 4595 |
| Growth factor independence I | BC032751 | 423 | 1268 |
| C-terminal end of the | BC018641 | 463 | 1388 |
| cds-elongation factor I alpha | | | |
| tRNA nucleotidyl transferase, | AF151805 | 435 | 1304 |
| CCA-adding 1 (TRNT1) | | | |
| Splicing factor, arginine/serine | M90104 | 222 | 665 |
| rich 2 | | | |
| Hypoxanthine | BC000578 | 219 | 656 |
| phosphoribosyltrasferase 1 | | | |
| Dyskerin | BC010015 | 515 | 1544 |
| Human stress-induced | BC002967 | 544 | 1631 |
| phosphoprotein 1 | | | |
| Hsp 70 like protein | AF112210 | 510 | 1529 |
| SH3 domain kinase binding | BC015806 | 666 | 1997 |
| protein 1 | | | |
| (SH3KBP1) | | | |
| Protein kinase C inhibitor 1 | HSU27143 | 127 | 381 |
| Pyruvate kinase | BC007952 | 532 | 1595 |
| Glutamate ammonia ligase | BC011852 | 374 | 1121 |
| (glutamine synthase) | | | |
| Galactosyltransferase I | BC007317 | 328 | 984 |
| DCAL-1 | NM_172004 | 167 | 505 |
| Down syndrome critical region 2 | AJ006291 | 289 | 867 |
| NS-associated protein 1 like | AF155568 | 563 | 1689 |
| protein | | | |
| MHC HLA class I cell surface | M32319 | 206 | 618 |
| antigen | | | |
| TCTP like protein | BC022436 | 139 | 417 |
| DHHC1 protein | AF441791 | 299 | 900 |
| Diazepam binding inhibitor | M14200 | 105 | 315 |
| HIRA interacting protein 5 | AJ132584 | 197 | 591 |
| TCAA3 (transforming acidic | AF093543 | 839 | 2517 |
| coiled-coil containing protein | | | |
| 3) | | | |
| Rad 50 interacting protein | AF317622 | 822 | 2466 |
| Structure specific recognition | BC005116 | 710 | 2130 |
| protein | | | |
| Macrophage galactose-type C- | XM_090246 | 140 | 420 |
| type lectin 2 like protein | | | |

6.2 Inhibition of GTP-GDP Exchange Activity of Arf1-GAP by TCTP

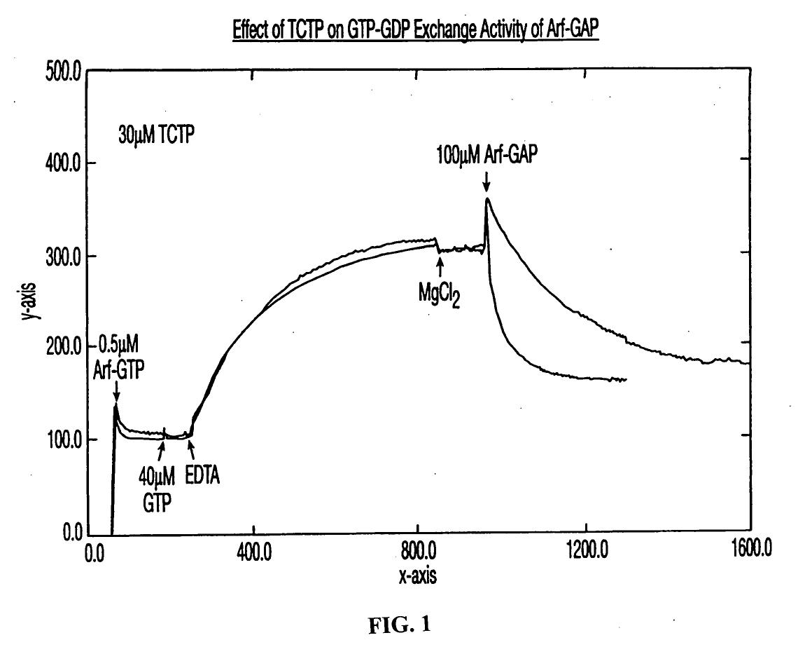

Into a buffer containing 1 mM free Mg²⁺, 30 uM TCTP and 0.5 uM [D17]ARF1 -GDP were added. The reaction was initiated by the addition of 40 uM GTP and 2 mM EDTA. To stop the reaction, 2 mM MgCl₂ was added. The Arf-GAP, at a concentration of 100 uM, was added. The reaction was measured in presence and absence of TCTP. The tryptophan fluorescence change that accompanies the conformational change of ARF1 from the inactive state (GDP-bound) to active state (GTP-bound) was followed in real-time to assess the activation of [D17]ARF1. As shown in FIG. 1, the results show that the function of Arf1, a binding partner of TCTP, is affected by the presence of TCTP. In particular, it is shown that TCTP inhibits the GTP-GDP exchange catalyzed by Arf1-GAP, implicating that Arf1 is kept in its active conformation in presence of TCTP.

6.3 Binding of TCTP with OP18 (Stathmin)

The binding between TCTP and its binding partner OP18 was characterized. OP18 is a phosphoprotein which is a substrate of kinases such as cdc2/cyclin B kinases, Ca⁺⁺/calmodulin dependent kinases, cAMP dependent protein kinases, and MAP kinases. Op 18 is also known to function as a microtubule destabilizing factor, inducing microtubule depolymerization. See Belmont et al., Trends in Biochem. Sci., 21: 197-198 (1996).

6.3.1 Materials and Methods

Time-lapse microscopy in TCTP overexpressing cells. HeLa cells were transfected with pEGFP-N1, pEYFP-N1-TCTPwt or pEYFP-N1-TCTPS46AS64A using calcium phosphate method. Five hours later, time-lapse movies were made for 72 hours using a Leica microscope equipped with a 20× lens and a CCD camera. Images were acquired every 10 minutes in phase contrast, complemented with fluorescent excitations every 12 hours in the aim to follow the transfected population of cells. The phenotypes were reported exclusively for fluorescent cells.

siRNA Experiments. HeLa cells were transfected twice by 24 hours with 50 nM siRNA TCTP (5′-AAGGTACCGAAAGCACAGTAA-3′) or 50 nM siRNA GFP (5′-AACACUGGUCACUACCUUCAC-3′) (Dharmacon research, Lafayette, Colo., USA) by using oligofactamine. Thirty six hours later, cells were fixed with 4% paraformaldehyde, and DNA was stained by DAPI. In time-lapse experiments, movies were made 24 hours after the double transfection for 72 hours, as described above. The phenotypes were studied on the overall population of cells. Western-blot analysis was done on total extract of cells lyzed in 20 mM Tris pH 7.5, 150 mM NaCl and 1% NP40 supplemented with protease inhibitors. TCTP and β-tubulin were detected with the polyclonal rabbit anti-TCTP antibody directed against the entire protein and the polyclonal rabbit anti-β-tubulin antibody (Santa-cruz H-235), respectively, and revealed with anti-rabbit HRP antibody.

Analysis of microtubule organization defects. HeLa stably expressing GFP-α tubulin were transfected with pDsRed2-TCTPS46AS64A. Forty eight hours later, cells were fixed with 4% paraformaldehyde, and DNA was stained by DAPI. Images from TCTP mutant expressing cells were acquired on the Olympus white field system described above, and then deconvoluated and reconstructed as previously described.

Yeast two-hybrid hunt. First 84 amino acids of TCTP were fused in-frame with the LexA DNA-binding domain of pEG202. A cDNA library derived from human monocytic leukemia U937 cells was cloned in galactose-inducible pYESTrp2 vector. A yeast two-hybrid hunt was performed substantially following the procedures described in Finley, R. J. et al., Oxford Universal Press, 169-203 (1995), incorporated herein by reference.

In vivo interaction. HeLa cells were lyzed for 1 hour in 1% Nonidet P-40 lysis buffer containing 25 mM Tris-HCl, pH 7.5, 150 mM NaCl, 10% glycerol, plus the protease inhibitors 1 mM 4-(2-aminoethyl)benzenesulfonyl fluoride (AEBSF), 1% aprotinin, 1 mM leupeptin, and 2 mM pepstatin (all reagents from ICN). Cell lysates were clarified by centrifugation at 14 000 rpm for 20 minutes. TCTP or OP18 were immunoprecipitated from lysates with either anti-TCTP (Cans et al., Proc. Natl. Acad. Sci. USA., 100: 13892-13897 (2003)) or anti-OP18 antibody (kindly provided by Dr M. Gulberg, Umea University, Sweden). The addition of G protein agarose beads (Amersham Biosciences) was followed for an additional 3 hours at 4° C. Immune complexes were washed four times in the lysis buffer, eluted in Laemmli buffer and analyzed by western-blot.

Transfection, preparation of native OP18 enriched extracts, and analysis of OP18 phosphorylation levels. 293 T cells were transfected by the standard calcium phosphate method, with the following constructs: pEYFP (Clontech, BD) (negative control), pEYFP-TCTP wide type (WT) or the mutant pEYFP-TCTP S46A/S64A. Twenty four hours after the transfection, the cells were treated with nocodazole (250 ng/ml) for 0 hour, 1 hour, 2 hours, 4 hours and 8 hours, and rinsed twice in PBS. The pellets were lyzed with 3-5 volumes of lysis buffer (50 mM Tris-HCl, pH 7.5, 250 mM NaCl, 10 mM EDTA, 1% Nonidet P-40, protease inhibitors cocktail and phosphatases inhibitors (10 mM sodium orthovanadate, 10 mM natrium fluoride (NAF)) on ice for 20 minutes, and centrifuged for 10 minutes at 14,000 rpm at 4° C. The supernatants were carefully removed and stored on ice while measuring their concentration of proteins by spectrophotometry at 595 nm (Bradford method). To enrich the concentration of OP18 in the extracts, 1.2 mg of each total extract was boiled at 75° C. for 7 minutes, put on ice for 1 minute, and centrifuged for 10 minutes at 14,000 rpm at 4° C. Then, the supernatants were precipitated in 5-6 volumes of 1% sucrose in methanol O/N at −20° C. The extracts were centrifuged for 30 minutes at 14,000 rpm at 4° C., and the pellets were dried under vacuum and resuspended in 40 μl of 2× native loading buffer (5× Tris-Glycine, pH 8.5, 50% glycerol, bromophenol blue). To analyze the expression levels of the different phosphorylated states of OP18, a native PAGE system that separates OP18 isoforms according to their charge was employed. Five μl of each native extract, corresponding to about 150 μg of total proteins, were loaded on native gels (250 mM Tris, pH 8,5, 6.25% acrylamide/bis, 0.1% ammonium persulfate (APS) and 0.08% TEMED), which were pre-run at 50 V for 30 minutes in 5× running buffer (765 mM Glycine, 100 mM Tris basis, pH 8.5). Then, the voltage was increased to 100 V. The separated proteins were transferred onto nitrocellulose membrane (Biorad), incubated with an antibody diluted 1/1000, recognizing the OP18 phosphorylated and unphosphorylated forms (kindly provided by Dr. M. Gulberg, Umea university, Umea, Sweden).

Preparation of soluble and polymerized tubulin. 293 T cells were transfected using the same conditions as described above. Twenty four hours after transfection, the cells were treated with nocodazole (250 ng/ml) for 0 hour, ½ hour, 2 hours, 4 hours and 6 hours. The treated cells were rinsed twice in PBS, lyzed on plates with 250 μl of a microtubule-stabilizing buffer (20 mM Tris-HCl, pH 6.8, 140 mM NaCl, 0.5 % Nonidet P-40, 1 mM MgCl₂, 2 mM EGTA, 4 μg/ml Taxol, protease inhibitors cocktail), scraped and transferred into a 1.5 ml microcentrifuge tube. Each plate was rinsed with a second 250 μl of lysis buffer, and the cell lysates were pooled. The lysis occurred for 20 minutes at room temperature. The microcentrifuge tubes containing 500 μl of lyzed cells contents were briefly mixed and then centrifuged at 14,000 rpm for 10 minutes at 4° C. Supernatants containing soluble tubulin were carefully separated from pellets containing polymerized tubulin and placed in a separate tube. The pellets were quickly rinsed with additional 20 μl of lysis buffer and resuspended in 125 μl of water. To each tube, was added 125 μl of 4× lysis buffer and 250 μl of 2× SDS dissociation buffer (Laemmli), in order to obtain 1× final concentration of salts comparable to the soluble fractions. The concentration of proteins in the supernatants was measured by spectrophotometry at 595 nm (Bradford method). Using these soluble tubulin concentration values, 60 μg of the polymerized tubulin fractions were run on a 10% acrylamide gel (SDS-PAGE system) and transferred on a nitrocellulose membrane (Biorad) that was incubated with an anti-a-tubulin (Sigma) diluted 1/2000.

In vitro tubulin polymerization assays. Pure bovine brain tubulin protein was reconstituted at a final concentration of 10 mg/ml in cold G-PEM buffer (80 mM Piperazine-N,N′-bis-[2-ethanesulfonic acid] equisodium salt (PIPES), 0.5 mM magnesium chloride (MgCl₂), 1 mM ethylene glycol-bis-(b-amino-ethyl ether)-N,N,N′,N′-tetra-acetic acid (EGTA), 1 mM Guanosine 5′-triphosphate (GTP), pH 6.9). Samples of 2 mg/ml tubulin in G-PEM buffer were incubated either with 3 μg of ovalbumin (negative control), with 3 μg of purified recombinant TCTP protein, or with 3 Mg of purified recombinant OP18 protein. Then, the samples were placed in quartz cuvettes and incubated at 32° C. Tubulin polymerization was observed by measuring the absorbance of the solution at 340 nm over time.

Immunofluorescence. HeLa cells, plated on coverslips (12 cm diameter), were washed twice with phosphate-buffered saline (PBS). Then, according to the type of antibodies used for the staining, the cells were fixed differently. For the TCTP/α-Tubulin and TCTP/OP18 staining, the cells were fixed with 4% paraformaldehyde in PBS for 20 minutes at room temperature, washed three times, incubated 5 minutes each in PBS, permeabilized with 0.1% triton in PBS for 10 minutes, and washed again. For the TCTP/γ-Tubulin staining, the cells were fixed/permeabilized with methanol for 10 minutes at −20° C. and washed with PBS. All of the following steps were done at room temperature. After saturation in 1% bovine serum albumin (BSA) in PBS for 15 minutes, cells were incubated with the primary antibody diluted with 1% BSA in PBS for 1 hour. The primary antibodies used were the goat polyclonal anti-OP18 (N-20) (Santa Cruz) diluted 1/1000, the rabbit polyclonal anti-p-OP18 (Ser 16)-R (Santa Cruz) diluted 1/1000, the chicken anti-TCTP (Cans et al., supra) diluted 1/4000, the mouse monoclonal anti-α-tubulin (Sigma) diluted 1/2000, and the mouse monoclonal anti-γ-tubulin (Sigma) diluted 1/2000. After washing in PBS (three times, 5 minutes each), the cells were incubated with the secondary antibody diluted in 1% BSA in PBS for 1 hour and washed again. As secondary antibodies (all from Molecular Probes), the followings were used: an anti-goat Alexa Fluor 594 diluted 1/4000; an anti-rabbit Alexa Fluor 594 diluted 1/4000; an anti-chicken Alexa Fluor 488 diluted 1/8000; and an anti-mouse Alexa Fluor 594 diluted 1/10000. For double staining, the procedures described above were sequentially repeated. Then, the cells were incubated with 1 μg/ml of Di Aminido Phenyl Indol (DAPI) in PBS for 5 minutes, washed with PBS twice (5 minutes each), and quickly washed once with water, before being mounted on slides in fluoromount-G (Southern Biotechnologies). Images were acquired with a wide field microscope (Olympus, Cell R), deconvolved with the Huygens software, and reconstructed in three dimensions with the Imaris bit plane software.

6.3.2 Results

6.3.2.1 Localization of TCTP During Mitosis

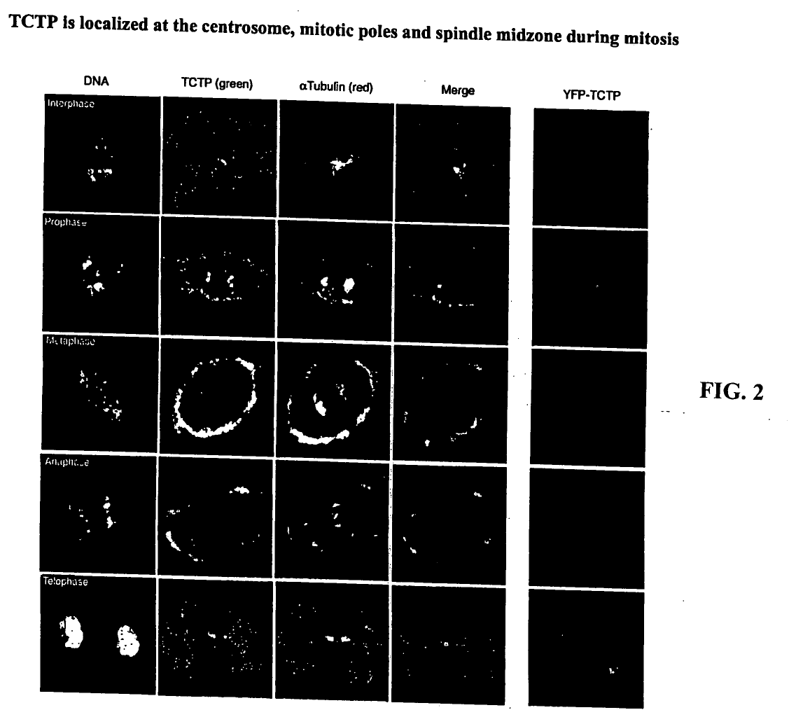

To better understand the function of TCTP, indirect immuno-fluorescence studies on HeLa cells were performed. A wide-field deconvolution microscopy was used to investigate the localization of the endogenous TCTP during the different phases of mitosis. FIG. 2 shows the staining of TCTP, α tubulin, and the nucleus using the DAPI as a dye. In interphase, the staining of TCTP was punctuated in the cytoplasm. Additionally, accumulation of TCTP at a particular structure in the cytoplasm was observed: the centrosome or microtubule organizing center (MTOC), the convergent point of the microtubule, as shown by the α tubulin staining and the merge of the two signals.

In prophase, TCTP stained the two centrosomes strongly. In metaphase, TCTP co-distributed with the poles of the microtubule spindle, as shown by the partial overlapping of a tubulin and TCTP staining. During Anaphase and Telophase, TCTP and a tubulin accumulated in the mid-zone and to the mid-bodies, respectively. The detection of the overexpressed TCTP fused to the Yellow Fluorescent Protein (TCTP-YFP) confirmed the presence of TCTP at the centrosome, the spindle pole and the mid-body during different phases of mitosis.

The results demonstrate that TCTP co-localized with centrosomal structure, the microtubule organizing center (MIOC), and could participate in the regulation of the cell cycle.

6.3.2.2 Induction of Mitotic Defects by TCTP

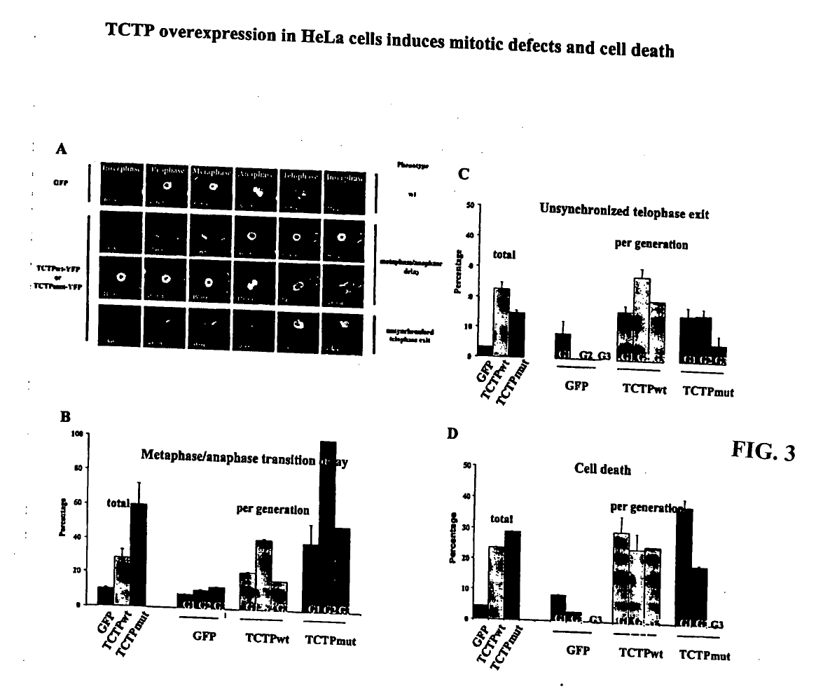

To determine whether TCTP could play a role in the progression of the cell cycle, the cell cycle of HeLa cells overexpressing fluorescent TCTP wild-type (TCTPwt-YFP), the polo-kinase mutant of TCTP (TCTPmut-YFP) or the control GFP, was analyzed using time-lapse microscopy. Cell divisions of transfected HeLa (G1, generation 1) and daughters cells (G2, generation 2; G3, generation 3) were observed for 72 hours by phase contrast (FIG. 3). In TCTPwt-YFP expressing cells and three independent experiments, a higher occurrence of pleiotropic defects was observed when compared to control cells. The defects were: metaphase/anaphase delay; unsynchronized telophase exit; and cell death.

Overexpression of both TCTPwt-YFP and TCTPmut-YFP induced a delay in the metaphase/anaphase transition of mitosis (FIG. 3A, middle panel). Indeed, while GFP positive cells entered into anaphase 50 minutes after the beginning of mitosis, TCTP positive cells entered into anaphase 120 minutes later. This phenotype was detected approximately 3 times and 6 times more frequently in TCTPwt-YFP or TCTPmut-YFP overexpressing cells, respectively, than in control cells (FIG. 3B, left panel). Moreover, this defect was stronger in both G2 (FIG. 3B, right panels), suggesting the accumulation of cellular defects during cell divisions. The strong metaphase/anaphase delay phenotype for the polo kinase mutant of TCTP indicated a regulation of TCTP activity by polo kinase. Finally, it was notable that this delay varied among the cells, suggesting a scale of cellular defects (data not shown), which may depend of the level of expression of YFP-TCTP in transfected cells.

In addition, TCTP overexpressing cells exhibited an unsynchronized telophase exit phenotype. As illustrated in FIG. 3A, bottom panel, daughters of the dividing cell were not synchronized at the beginning of interphase, one staying round and the other one adhered. This phenotype was found approximately 5 times more frequently in TCTPmut-YFP expressing cells than in the control, and 7 times more frequently when TCTPwt-YFP was used. Moreover, a stronger effect on the G2 population was also observed (FIG. 3C).

TCTP overexpressing cells also had a higher cell death rate in interphase than GFP positive cells, i.e., approximately 5 times more frequently, in the three generations.

The metaphase/anaphase transition is a highly regulated cell cycle checkpoint controlled by the anaphase promoting complex. This complex ensures the fidelity of the chromosomes segregation at the metaphase/anaphase transition by checking the anchoring of microtubules to kinetochores of chromosomes and by delaying mitotic progression to allow correction of defects. See Musacchio et al., Nat. Rev. Mol. Cell Biol., 3: 731-741(2002).

The results show that TCTP co-localized with the microtubule organizing center of cell and promotes similar effects as polo kinase on the cell cycle. Hence, the overexpression of TCTP may perturb the microtubule network organization and/or play a role in the metaphase/anaphase checkpoint.

6.3.2.3 Effects of TCTP on Spindle Organization

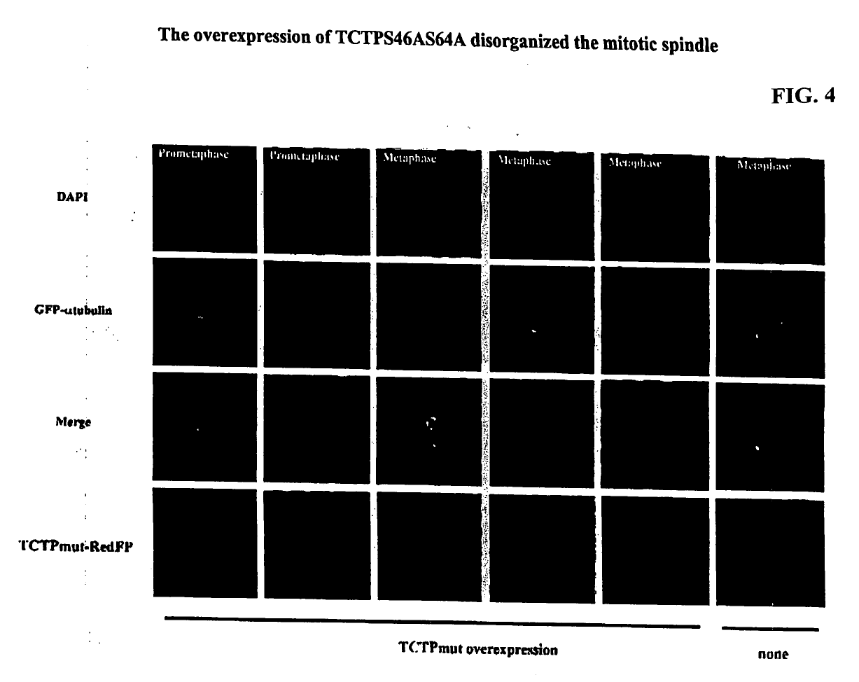

To determine whether TCTP overexpression induces a deficient spindle organization, stable HeLa cells expressing GFP-a-tubulin were used to visualize the spindle organization in pro-metaphase and metaphase. Chromosomes were stained with DAPI to observe their condensation. Since TCTPmut-YFP exhibited the strongest metaphase/anaphase delay phenotype, this particular mutant protein fused to the Red-FP tag in stable cells were overexpressed in the cells. FIG. 4 shows the metaphasic spindle of TCTP non-overexpressing cells (right column), compared to microtubules organization of TCTP overexpressing cells (left columns). The majority of TCTP positive cells presented a deficient spindle organization with pleiotropic effects, indicative of unorganized spindle, asymmetric spindle, multipolar cells and defects in the chromosome alignment in metaphase. These data supporting mitotic defects observed in time-lapse microscopy experiments confirmed that TCTP plays a role in the spindle formation and organization.

To precisely determine the nature of the defects, whether TCTP impairs microtubules stability and/or a correct microtubules/chromosomes anchorage may be examined. Anti-Mad2 antibody may be used as a marker of the microtubules/kinetochores anchoring in immunofluorescence experiments.

6.3.2.4 Effects of TCTP Downexpression on Mitosis

The previous experiments demonstrated that the modification of TCTP expression level promoted cell cycle progression. To confirm these data, TCTP expression in HeLa cells was knocked down using small interference RNA (siRNA). As shown in FIG. SA, an equilibrated loading of total extract (western blot anti-β tubulin) showed that siRNA directed against TCTP induced a decrease in the level of TCTP expression compared to the siRNA control directed against GFP.

First, transfected cells were fixed with paraformaldehyde. DNA was stained with DAPI and mitotic phases of dividing cells were determined. It was observed that in siRNA TCTP transfected cells, more abundant population of cells were in telophase as compared to the control cells.

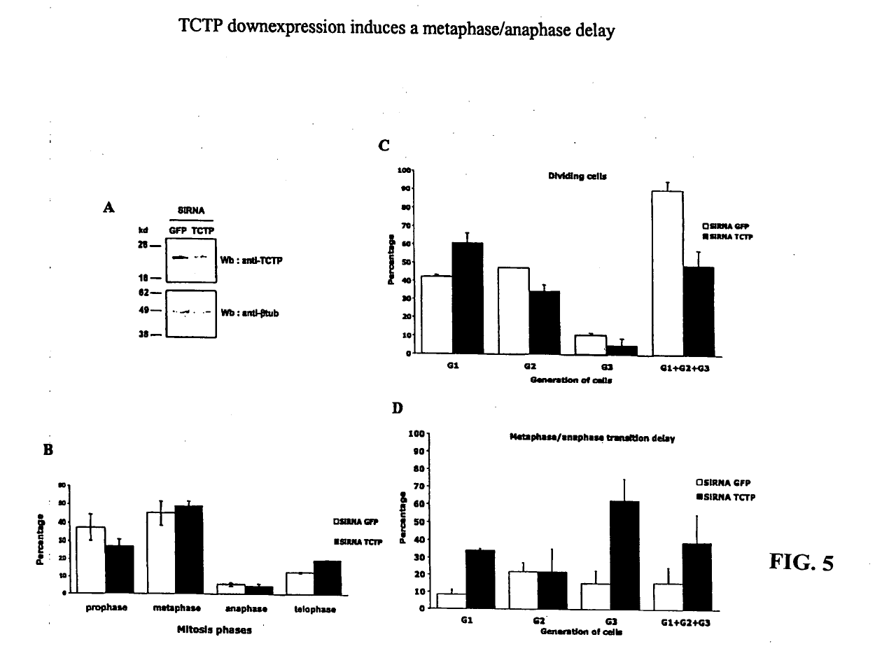

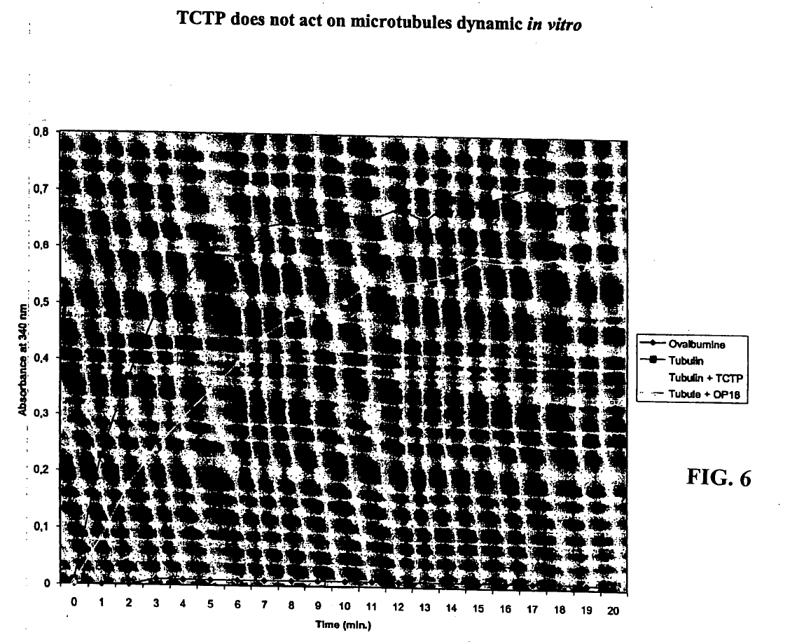

Second, time-lapse microscopy experiments were performed for 72 hours after a double siRNA transfection, and the division of the whole population of cells was followed. Two major phenotypes were observed. siRNA TCTP transfected cells presented a higher propensity to die during the transfection time, correlated with the decrease of the division rate of siRNA TCTP transfected population (FIG. 5C), when compared to control cells (data not shown). Further, this population exhibited approximately 2.5 times higher metaphase/anaphase transition delay phenotype than the control cells. Notably, the major effect was observed in G3, suggesting a cumulative effect during the cell division.

Overall, these data demonstrated that TCTP expression level and TCTP phosphorylated state were important in the progression of the cell cycle, especially for the organization of the mitotic spindle.

6.3.2.5 Effect of TCTP on Microtubules Dynamic In Vitro

Since TCTP exerts tubulin and microtubules binding properties (Gachet et al., J. Cell Sci., 1 12(Pt 8): 1257-1271 (1999)), whether TCTP could directly regulate the microtubule dynamics was investigated. An in vitro tubulin polymerization assay, using purified tubulin, was developed. As shown in FIG. 6, the polymerization of the purified tubulin was followed by the increase of the absorbance at 340 nm over a period of time. In the presence of 3 μg of purified OP18 from E. Coil, the polymerization of tubulin was abolished. The addition of 3 μg of bacterially expressed and purified TCTP on purified tubulin did not appear to affect the polymerization rate. The data suggests that TCTP does not influence the microtubule dynamics in vitro.

6.3.2.6 Interaction of TCTP with OP18 In Vivo

A yeast two-hybrid hunt was performed to identify TCTP-interacting proteins, which may play a role in directly regulating the dynamics of microtubule. The first 84 amino acids of TCTP were used as bait to screen a cDNA library obtained from the human monocytic leukemia U937 cell line. OP18/stathrnin, a microtubule destabilizing molecule, was identified as a TCTP NH₂-termunus end binding protein.

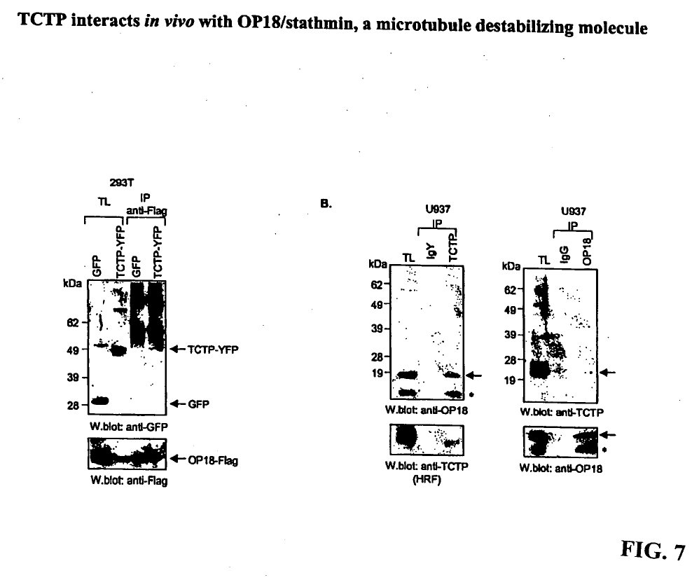

In order to confirm that the interaction occurs in vivo, co-immunoprecipitation experiments were performed. First, as shown in FIG. 7A, TCTPwt-YFP cells were compared to YFP negative control cells. In the TCTP overexpressing cells, TCTP was detected in complex with OP18, Flag tagged and immunoprecipitated with anti-Flag antibody. To validate the interaction, whether the endogenous TCTP and OP18 proteins interact with each other was examined using reciprocal co-immunoprecipitation on U937 cell lysate. Western-blot analysis revealed that endogenous OP18 is detected only in the immunoprecipitates obtained using a chicken specific anti-TCTP antibodies, compared to an isotype-matched IgY control antibody. Reciprocally, endogenous TCTP was present only in an immune complex, immunoprecipitated with a goat specific anti-OP18 antibody compared to a non specific IgG, used as control. These data show that endogenous TCTP interact with OP18.

6.3.2.7 Mapping of the Interacting Region in OP18

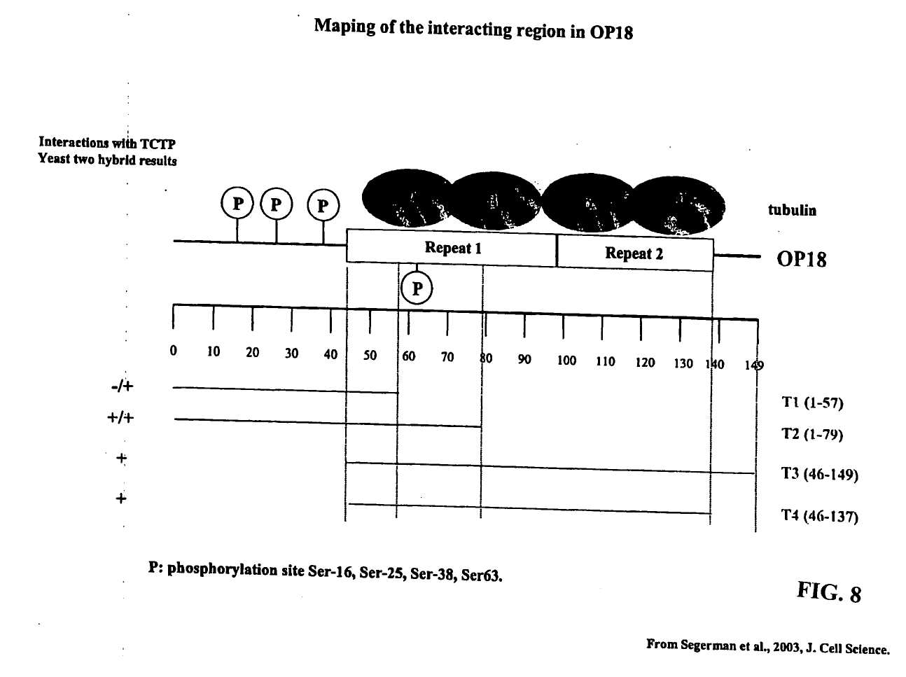

For further characterization, truncated mutants of OP18 were investigated in order to determine the region of OP18 that interacts with TCTP. As depicted in FIG. 8, OP18 is composed of a NH₂-terminus end from the residues 1 to 46, where the phosphorylation sites (P) are indicated on Serines 16, 25, and 38, and a long helical structure encompassing the residues 46 to 149, which contains two tubulin heterodimer binding sites. Four truncated mutants of OP18 were designed: T1, encompassing residues 1 to 57; T2, encompassing residues 1 to 79; T3, encompassing residues 46 to 149; and T4, encompassing residues 46 to 137. The truncated mutants were cloned in a two hybrid yeast vector and screened for their ability to bind TCTP. The results showed that TI does not interact with TCTP, whereas T2, T3, and T4 bind to TCTP. In particular, it was found that T2 interacts better with TCTP than T3 or T4. Therefore, it was determined that the region of OP18 encompassing the residues 46 to 79 may be the minimal region of OP18 required for its interaction with TCTP.

6.3.2.8 Co-Localization of TCTP and OP18 During Mitosis

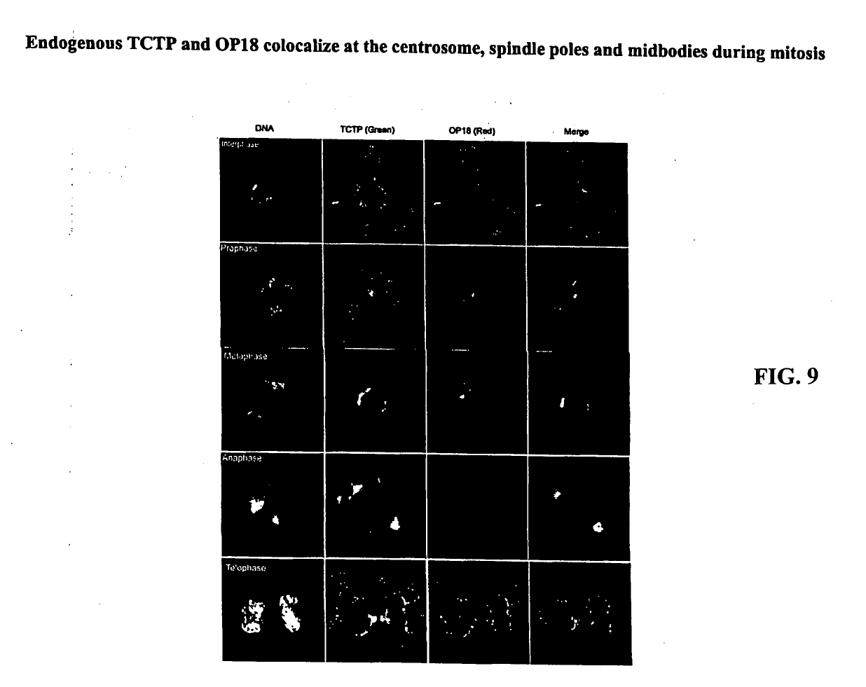

Whether OP 18 is co-distributed with TCTP was investigated using wide-field deconvolution microscopy technique, substantially similar to the one used for the characterization of the localization of TCTP during mitosis. Indirect immuno-fluorescence experiments using a chicken specific anti-TCTP antibodies and a rabbit anti-OP18 antibodies that recognizes specifically its phosphorylated forms were performed, since OP18 is highly phosphorylated in mitosis. As shown in FIG. 9, TCTP and OP18 exhibited the same localization pattern, both localized at the centrosomes, spindle pole and mid-bodies during the prophase, metaphase, anaphase and telophase, respectively, as seen on the merge panel where the signal reflect the presence of TCTP and OP18.

6.3.2.9 Modulation of OP18 Phosphorylation by TCTP

Since TCTP interacts with OP18, whether the binding of TCTP could change the phosphorylation state of OP 18 was investigated to confirm the regulation of the OP1 8 activity by TCTP. To monitor endogenous OP18 phosphorylation state, native gels on which the different degree of phosphorylation can be distinguished were used. See Marklund et al., J. Biol. Chem., 268: 15039-15047 (1993a), incorporated herein by reference. In short, the native gel technique allows the separation of the molecule according to their charge. Therefore, the more negative charges the protein has (e.g., by attachment of phosphate groups), the further it migrates in the gel. Immuno-blotting technique with a specific anti-OP18 antibody (gift from M. Gullberg, Umea, Sweden) was used to reveal the different phosphorylated OP18 species. In order to see the apparition of OP18 phosphorylation, the 293 HEK cells were treated with nocodazole for different periods of time. Nocodazole, an anti-mitotic agent that binds to microtubules and depolymerize them, induces stress to cells, blocking the division cycle at pro-metaphase state and triggering OP18 phosphorylation.

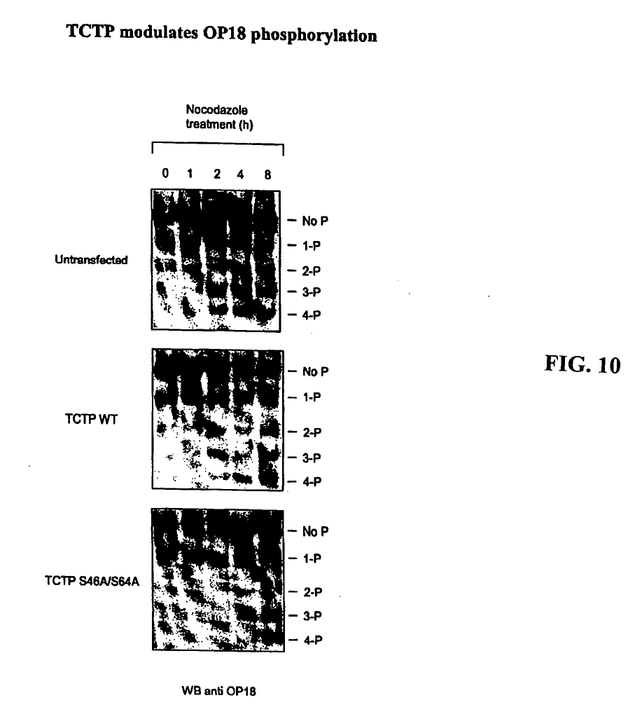

As shown in FIG. 10, upper panel, when the cells were not treated with nocodazole, OP18 was not phosphorylated (No-P) or one residue weakly phosphorylated (1-P). After the nocodazole treatment, the progressive apparition of the highly phosphorylated forms of OP18 was observed. OP18 containing three phosphorylated sites (3-P) and four phosphorylated sites (4-P) were detectable from 2 hours of treatment.

TCTPwt-YFP or TCTPmut-YFP was then overexpressed to examine whether TCTP modulates the phosphorylation state of OP18. As shown in FIG. 10, middle panel, highly phosphorylated species (3-P and 4-P) of OP18 were little detected after 4 hours of treatment. The presence of the highly phosphorylated species was also much less at earlier time point compared to the control. The results suggest that overexpression of TCTP WT result in the reduction of the level of OP18 phosphorylation.

As shown in FIG. 10, lower panel, when TCTPmut-YFP was overexpressed, the OP18 phosphorylated forms were not detectable even prior to 4 hours of treatment. Altogether these data suggest that TCTP may be a negative regulator of OP18 phosphorylation state. This finding is in agreement with the finding that TCTP binds to OP18, in particular, to residues 46 to 79 (see FIG. 8), which could influence the phosphorylation state of OP18 since it could “mask” certain phosphorylation site (such as Ser-63).

6.3.2.10 Induction of Microtubule Depolymerization by TCTP

Since TCTP overexpression lead to a decrease of OP18 phosphorylation, and consequently, contribute to the maintenance of OP18 in its active state, it was hypothesized that TCTP overexpression would induce the microtubule depolymerization. To confirm this hypothesis, an assay that enables the isolation and monitoring of the tubulin in a microtubule polymerized state only, but not in a free soluble state, was performed. Briefly, after lysis of cells in a hypotonic buffer containing low dose of Taxol (a microtubule stabilizing agent) and a centrifugation, the polymerized fraction of tubulin was preserved and purified. Then, the amount of polymerized tubulin in cells was monitored by western blot using an anti-α-tubulin antibody.

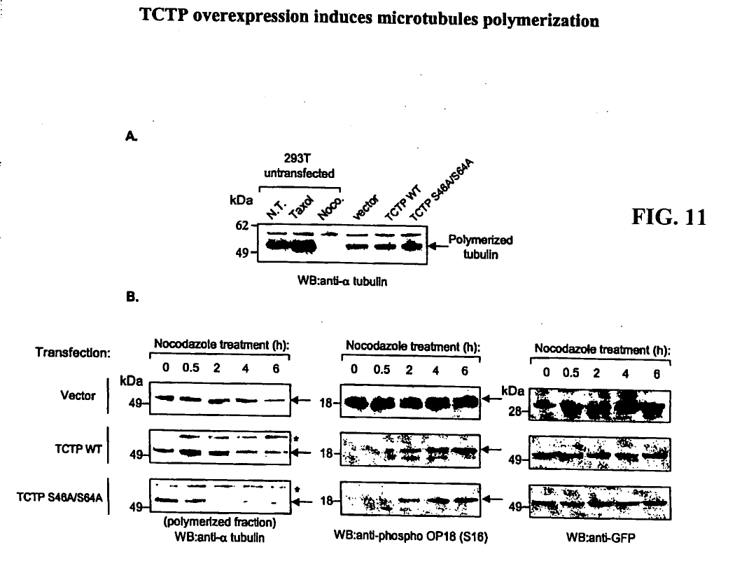

As shown in FIG. 11A, when the 293T HEK cells were treated 16 hours with 100 nM Taxol, a higher amount of polymerized tubulin was detected as compared to the untreated cells. In addition, after 16 hours of nocodazole treatment, a microtubule depolymerization agent, no polymerized tubulin was present. These data confirmed that only the polymerized tubulin fraction, but not the free soluble fraction, was being analyzed.

To address whether TCTP induced depolymerization of microtubule, TCTPwt-YFP or TCTPmut-YFP was overexpressed in 293T HEK cells, and the extent of tubulin polymerization after treatment of the cells with nocodazole was monitored. As shown in FIG. 11B, left panels, when the cells transfected with a vector control were incubated with nocodazole in increasing period of time, tubulin polymerization decreased proportionally to the time length of treatment. In TCTPwt-YFP overexpressing cells, the decrease in tubulin polymerization was quicker than that in the control cells. Moreover, TCTPmut-YFP overexpression led to an even higher decrease in the polymerized fraction. These data show that the overexpression of TCTPwt-YFP induces the depolymerization of microtubule and that TCTPmut-YFP promotes a greater effect on the microtubule dynamics.

Moreover, as shown in FIG. 11B, middle panel, using a phospho-specific anti-OP18 antibody, the finding that the overexpression of TCTPwt-YFP or TCTPmut-YFP (see FIG. 10) induces a decrease in OP18 phosphorylation after a nocodazole treatment was also confirmed. These results suggest that TCTP induces a diminution of OP18 phosphorylation, keeping OP18 active, concomitantly increasing the rate of microtubule depolymerization.

6.4 Binding of TCTP With eEF1A and eEF1Bβ

The binding between TCTP and its binding partners eEF1A and eEF1Bβ, eukaryotic elongation factors, was characterized.

6.4.1 Materials and Methods

Antibodies. Rabbit anti-eEF1Bβ and chicken anti-TCTP antibodies were generated against synthetic peptides corresponding to residues 14-30 of human eEF1Bβ or residues 55-65 of human TCTP, respectively (Agro-Bio, La Ferté St Aubin-France). Rabbit anti-TCTP and mouse anti-eEF1A antibodies were purchased from M.B.L. (Nagoya, Japan) and Upstate Biotechnology, respectively.

Purification of recombinant proteins. Full-length TCTP, eEF1Bβ and NKTR cDNAs were cloned in-frame into pGEX-6P (Amersham Bioscience). GST fusion proteins in BL21DE3 bacteria strain (Stratagene) were expressed by the addition of 0.1 mM IPTG for 16 hours at room temperature. Cells were lyzed for 30 minutes in a 1% NP40 buffer [10 mM Tris-HCl pH 7.5, 150 mM NaCl, 10% glycerol, 1 mM EDTA plus the protease inhibitors 1 mM AEBSF, 1% aprotinin, 1 mM leupeptin and 2 mM pepstatin (all reagents from ICN)]. The lysates were clarified by sonication and centrifugation (14,000 rpm) for 30 minutes at 4° C. and, GST fusion proteins were purified with glutathione-sepharose beads (Amersham Bioscience). The beads were washed three times with the 1% NP40 buffer, and the quality of the purification was determined by Coomassie Blue staining. The GST fusion proteins immobilized on beads were either incubated with IVT or purified eEF1A. For nucleotide exchange reaction experiments, TCTP and eEF1Bβ were recovered from the beads after the removal of the GST moiety by adding the Prescission protease according to the manufacturer's instructions (Amersham Bioscience). The purified proteins were dialysed and stored at −20° C. in a 50% glycerol solution containing 50 mM Tris-HCl (pH 7.5), 100 mM KCl, 1 mM DTT and 0.1% Triton X-100.

Yeast two-hybrid. Full-length or the first 84 amino acids of TCTP were fused in-frame with the LexA DNA binding domain of pEG202. A cDNA library derived from human monocytic leukemia U937 cells was cloned into galactose-inducible pYESTrp2 vector. Yeast two-hybrid hunt was performed as described in Finley, R. J. et al., Oxford Universal Press, 169-203 (1995), incorporated herein by reference.

In vitro and in vivo interaction. In vitro transcribed/translated ³⁵S-methionine labeled proteins were generated following the manufacturer's description (Promega). GST or GST-fusion proteins immobilized on beads were incubated with IVT radiolabeled products or purified rabbit eEF1A for 3 hours at 4° C. Proteins bound to the GST or GST alone, were washed and eluted directly in Laemmli buffer or in presence of 10 mM glutathione (ICN) (which is published as supporting information on the PNAS website). For detection of endogenous interactions, 293T and HeLa cells were lyzed for 1 hour in 1% NP40 lysis buffer containing 25 mM Tris-HCl pH 7.5, 150 mM NaCl, 10% glycerol, plus the protease inhibitors ImM AEBSF, 1% aprotinin, 1 mM leupeptin and 2 mM pepstatin (all reagents ICN), and cell lysates clarified by centrifugation (14,000 rpm) for 20 minutes. Endogenous TCTP or eEF1Bβ were immunoprecipitated from lysates with either anti-TCTP, anti-eEF1Bβ or an isotype-matched control antibody. The addition of protein G-agarose beads (Amersham Bioscience) was followed for an additional 3 hours at 4° C. Immune-complexes were washed 4 times in the lysis buffer, eluted in Laemmli buffer, and analyzed by western blot.

Immunofluorescence analysis. HeLa cells plated on glass coverslips were fixed in 4% paraformaldehyde/phosphate buffer saline (PBS) solution for 20 minutes at 4° C. After 3 washes with PBS, cells were permeabilized at room temperature with 0.25% Triton-X 100. Cells were washed and incubated for 30 minutes in blocking buffer [PBS containing 1% bovine serum albumin (BSA) (Sigma)]. Primary and secondary antibodies diluted in PBS-1% BSA were sequentially added for 1 hour at room temperature, followed by 3 washes in PBS. Cells were quickly rinsed in water and coverslips mounted on a slide. The secondary antibodies used in this study included anti-mouse or anti-rabbit antibodies conjugated to either AlexaFluor488 or AlexaFluor594 and were purchased from Molecular Probes (Eugene, Oreg.). The anti-chicken antibodies were either conjugated to FITC (Dako) or CY3 (Jackson laboratories). Confocal imaging was performed on a Leica TCS SP1 confocal microscope.

Affinity chromatography and mass spectrometry analysis. Five miligrams of human purified recombinant TCTP were crosslinked to a HI-TRAP NHS activated column (Pharmacia) according to the manufacturer's instructions. Three hundreds mg of total cell lysates from HEK 293T were loaded on the described TCTP affinity column. The column was preequilibrated with 10 volumes of 10 mM Tris-HCl (pH 8) at 4° C. with a flow rate of 0.3 ml/min, washed with 10 volumes of 100 mM Tris-HCl (pH 8) and 10 volumes of 10 mM Tris-HCl (pH 8). After a stable baseline was reached, the elution was done with 6 volumes of 50 mM glycine (pH 3). The eluate was immediately neutralized with 1M Tris-HCl (1/10 volume). The collected fractions were concentrated approximately 80 fold using an Amicon ultra-4 5000 MWCO (Millipore) and subjected to SDS-PAGE. Protein bands were visualized by Coomassie G250 (Biorad) staining, excised and in-gel-digested with trypsin (Cavusoglu et al., Proteomics, 3: 217-223 (2003), incorporated herein by reference). Peptide extracts (0.5 μl) were mixed with an equal volume of saturated alpha-cyano-4-hydroxycinnamic acid (LaserBio Labs) in 50% acetonitrile and applied to the target. Mass measurements were carried out on a Bruker Reflex IV MALDI-TOF spectrometer in the positive-ion reflector mode. The acquisition mass range was 850-3500 Da with low mass gate set at 700 Da. Internal calibration was performed using autolytic trypsin peptides (m/z=842.51, 2211.11 and 2807.47 Da). Mono-isotopic peptide masses were assigned manually using the Bruker X-TOF software. Database searches were performed using Profound program (http://prowl.rockefeller.edu/) with the following values: protein molecular mass between 30-100 Da, trypsin digestion with one missed cleavage allowed, cysteines modified by carbamidomethylation and methionine oxidation.

Guanine nucleotide exchange assay. Rabbit liver eEF1A was purified as described in Shalak et al., Ukr Biokhim Zh, 69: 104-9 (1997), incorporated herein by reference. The guanine nucleotide exchange on eEF1A was monitored as described in Negrutskii et al., J Biol Chem, 274: 4545-50 (1999) and Bec et al., J Biol Chem, 269: 2086-92 (1994), both of which are incorporated herein by reference. The eEF1A-[³H]GDP complex was prepared following incubation of 4 μM eEF1A with 4 μM [³H]GDP (Amersham Biosciences; 1500 Ci/mol) in 80 μl of 45 mM Tris-HCl (pH 7.5) containing 0.5 mM DTT, 10 mM magnesium acetate, 100 mM NH₄Cl, 1 mg/ml bovine serum albumin and 25% glycerol for 5 minutes at 37° C. The reaction mixture was placed on ice and diluted by addition of 640 μl of ice-cold exchange buffer [20 mM Tris-HCl (pH 7.5), 10 mM magnesium acetate, 50 mM NH₄Cl, 10% glycerol]. The exchange reaction was conducted at 0° C. after addition of 160 μl of exchange buffer containing nucleotide and specified exchange factors. Aliquots of 100 μl were taken at times indicated and immediately filtered through nitrocellulose filters (Millipore; pore size 0.45 μm). Filters were washed 3 times with 1 ml of ice-cold washing buffer [20 mM Tris-HCl (pH 7.5), 10 mM magnesium acetate, 100 mM NH₄Cl, 0.1 mg/ml bovine serum albumin], dried and counted in a liquid scintillator.

6.4.2 Results

6.4.2.1 Interaction Between TCTP and eEF1A and eEF1B

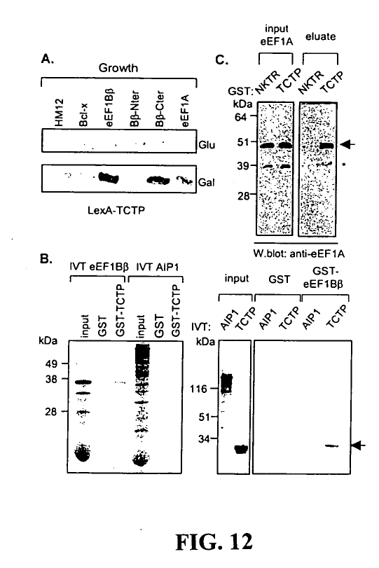

A yeast two hybrid hunt was undertaken to identify proteins that interact with TCTP. Full-length or the first 84 amino acids of TCTP were used as bait to screen a cDNA library obtained from the human monocytic leukemia U937 cell line. Among the positive clones isolated were two proteins involved in the elongation step of protein synthesis, the GTPase eEF1A and one of its guanine exchange factors, eEF1Bβ. Mating assays subsequently confirmed an interaction between LexA-TCTP and either B42-eEF1A or B42-eEF1Bβ (Table 2; FIG. 12A).

As defined by growth and β-gal activity, a robust interaction between LexA-TCTP and B42-eEF1Bβ was observed. A LexA-TCTP and B42-eEF1A interaction was also seen, less strong. Furthermore, the C-terminal GEF-containing region of eEF1Bβ (amino acids 153-281) was mapped as the TCTP binding region (FIG. 12A).

GST pull down assays confirmed direct and reciprocal binding between TCTP and eEF1Bβ. In vitro transcribed/translated (IVT) ³⁵S-labelled eEF1Bβ derived from reticulocyte lysates bound to GST-TCTP (FIG. 12B; left panel), but not to GST alone. Furthermore, GST-TCTP did not interact with the IVT negative control protein ALG-2 interacting protein 1 (AIP1). A reciprocal interaction was also demonstrated for GST-eEFBβ and IVT-generated TCTP (FIG. 12B; right panel).

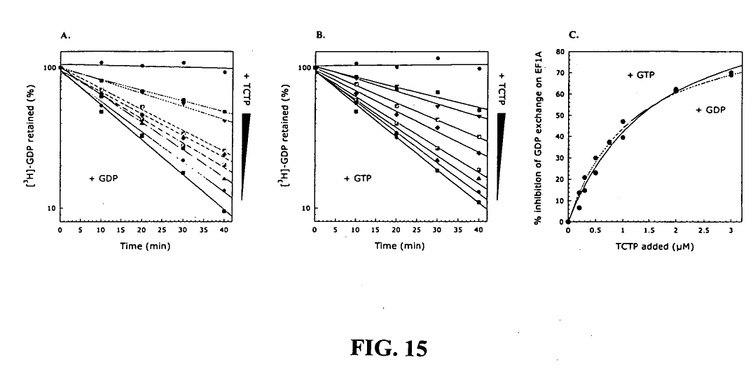

Moreover, purified eEF1A derived from rabbit liver bound specifically to GST-TCTP but not to the negative control, GST-NKTR (NK tumor recognition protein-1) (FIG. 12C). Because the apparent K_{d} of TCTP-eEF1A interaction is high (see FIG. 15C), the binding of TCTP to eEF1A in a nucleotide dependent manner in an in vitro pull down experiment could not be quantitatively addressed. Kinetic studies showed that TCTP preferentially binds the GDP form of the factor.

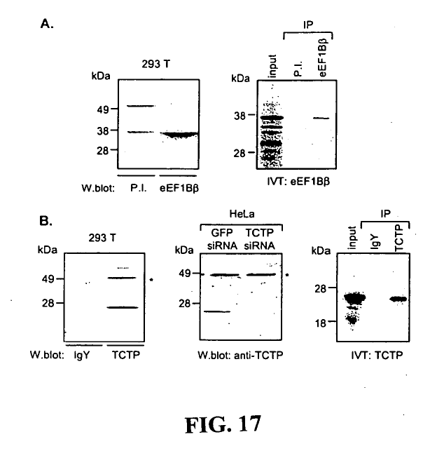

To investigate the presence of endogenous interaction between TCTP and eEF1Bβ, antibodies directed against TCTP and eEF1Bβ were generated and initially tested on total cell lysates derived from 293T cells immunoblot analysis revealed that the anti-TCTP antibody detected a protein band of 23 kDa, corresponding to its expected molecular weight (FIG. 17). In addition, the anti-eEF1Bβ antibody recognized a single protein band of 36-38 kDa, which corresponded to the predicted size of eEF1Bβ. Finally, anti-TCTP and anti-eEF1Bβ antibodies immunoprecipitated their respective recombinant protein, indicating that both antibodies recognize native proteins (FIG. 17).

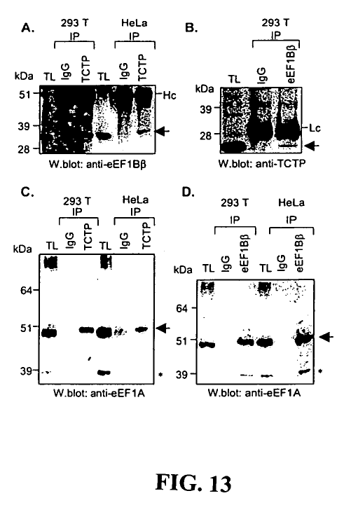

Co-immunoprecipitation experiments were subsequently carried out on lysates derived from either 293T or HeLa cells to identify the presence of an endogenous association between TCTP with either eEF1Bβ or eEF1A. Anti-rabbit TCTP or isotype-matched IgG control antibodies were initially incubated with cell lysates. Immunoblot analysis with antibodies against either eEF1Bβ (FIG. 13A) or eEF1A (FIG. 13C) revealed that anti-TCTP but not rabbit IgG specifically co-immunoprecipitated protein bands of 36 kDa and 51 kDa, respectively. In addition, immunoblotting with the anti-chicken TCTP antibody on immune-complexes immunoprecipitated with anti-eEF1Bβ revealed a reciprocal association between TCTP and eEF1Bβ (FIG. 13B). FIG. 13D illustrates an association between eEF1Bβ and eEF1A. Finally, mass spectrometric analysis of affinity purified TCTP binding partners confirmed that eEF1A interacts with TCTP. eEF2, a GTPase also involved in elongation was identified as an additional TCTP interacting partner in the same screening (Tables 3 and 4). Overall, the results showed that TCTP associates with eEF1Bβ and eEF1A, proteins involved in translation elongation.

6.4.2.2 Co-Localization of TCTP with eEF1A or eEF1B

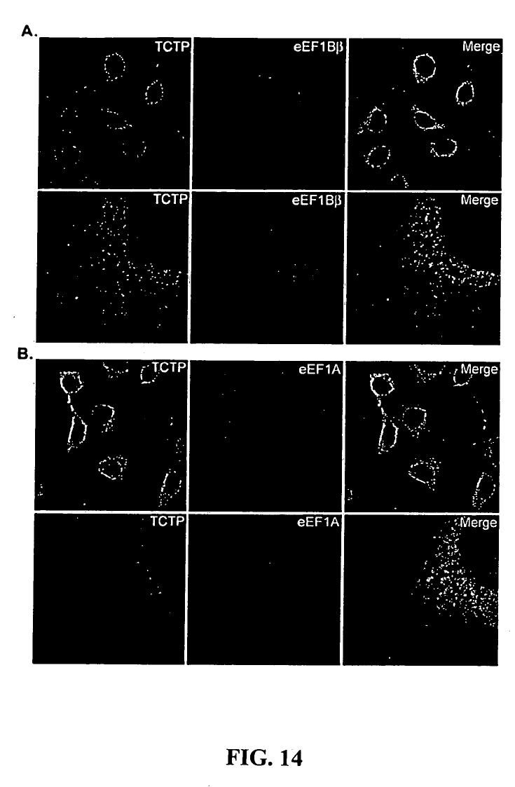

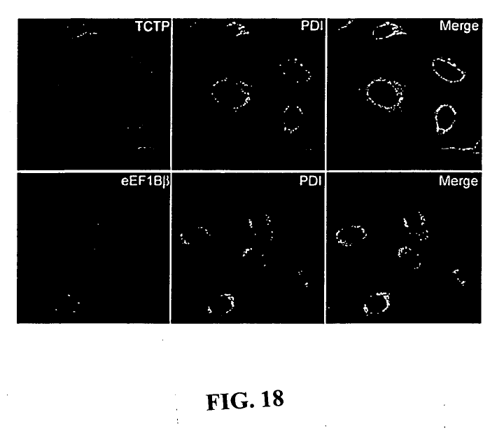

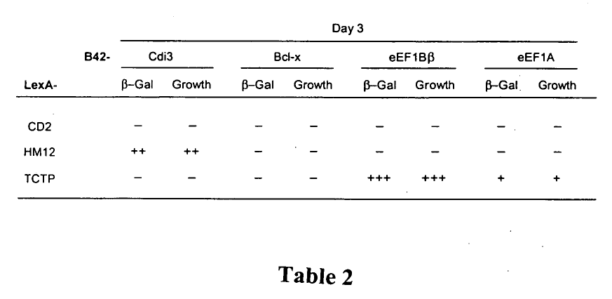

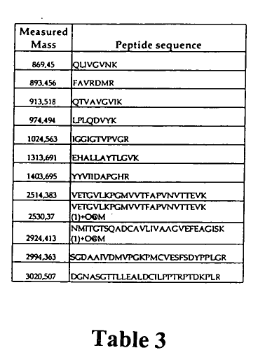

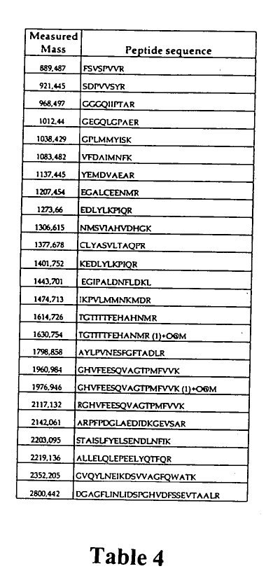

Indirect immunofluorescence studies were performed on HeLa cells to further investigate an endogenous association between TCTP and either eEF1Bβ or eEF1A. FIG. 14 shows that staining with the anti-chicken TCTP antibody appeared punctuate and cytoplasmic that partially co-localized with the endoplasmic reticulum marker Protein Disulfide Isomerase (PDI) (FIG. 18). Confocal imaging also showed a strong perinuclear staining with the anti-TCTP, partially overlapping with ER marker. Anti-eEF1Bβ staining was restricted primarily to the ER (FIG. 14A, upper panel), co-staining with PDI (FIG. 18). The overlay of TCTP and eEF1Bβ staining indicated that these proteins partially co-localize to the perinuclear region of the cell.

In addition, co-localization studies of eEF1A and TCTP were also performed. Immunofluorescence analysis using an anti-eEF1A antibody revealed staining around the nucleus and throughout the cytoplasm. Confocal analysis on HeLa cells stained with anti-chicken TCTP and anti-eEF1A antibodies revealed a partial co-localization around the nucleus (FIG. 14B).

6.4.2.3 Stabilization of GDP Form of eEF1A by TCTP

The functional relevance of a TCTP and eEF1A association was further investigated by monitoring the effects of TCTP on the rate of dissociation of GDP from the eEF1A-[³H]-GDP complex. Whether TCTP preferentially binds to the GDP-bound form of the factor (and pushes the equilibrium toward the formation of eEF1A-GDP) or associates with the nucleotide free form of eEF1A (and displaces the exchange reaction toward the formation of eEF1A) was determined.

When eEF1A was preloaded with [³H]-GDP and incubated in the presence of saturating amounts of unlabeled GDP (150 μM), GDP dissociation followed monoexponential kinetics corresponding to a half-life of the complex of 12 minutes (FIG. 15A). This corresponded to a rate of 0.082±0.004 pmol GDP exchanged/min/pmol of eEF1A. In the absence of free GDP in the incubation mixture, the eEF1A-[³H]-GDP complex remained stable over a period of 40 minutes. Increasing amounts of TCTP (0.2 μM to 3.0 μM) were added in the exchange assay and the rate of dissociation of the eEF1A-[³H]-GDP complex was monitored in the presence of 150 μM free GDP (FIG. 15A). TCTP by itself did not bind nucleotides. When 3 μM of TCTP were added, the half-life of the eEF1A-[³H]-GDP complex increased to 39 minutes, corresponding to a rate of GDP dissociation of 0.026±0.003 pmol GDP exchanged/min/pmol of eEF1A.

The data showed that TCTP is devoid of exchange activity; its addition decreases the rate of GDP exchange on eEF1A. The inhibition of GDP exchange by TCTP was concentration dependent and followed a saturation kinetics with an apparent dissociation constant K_{d} of 1.2±0.2 μM (FIG. 15C). The stabilization of the eEF1A-[³H]-GDP complex by TCTP suggests that TCTP preferentially binds the GDP form of eEF1A as compared with the nucleotide free form of the factor.

The eukaryotic elongation factor eEF1A binds GDP and GTP with similar affinity (2-4 μM). To determine whether TCTP preferentially binds the GDP or the GTP form of eEF1A, the effect of the addition of TCTP on the GDP-GTP exchange on eEF1A was monitored (FIG. 15B). When unlabeled free GTP (150 μM) was added in the exchange reaction instead of GDP, a similar protection of the eEF1A-[³H]-GDP complex was observed (with the rate of GDP dissociation decreasing from 0.078±0.004 to 0.024±0.003 pmol GDP exchanged/min/pmol of eEF1A), showing that TCTP does not displace the equilibrium towards the formation of an eEF1A-GTP complex. The apparent K_{d} of TCTP for the GDP form of eEF1A (1.6±0.3 μM, FIG. 15C) was not significantly affected. These results show that TCTP preferentially binds the GDP form of eEF1A and impairs GDP dissociation.

6.4.2.4 Inhibition of eEF1Bβ-mediated Exchange Reaction by TCTP

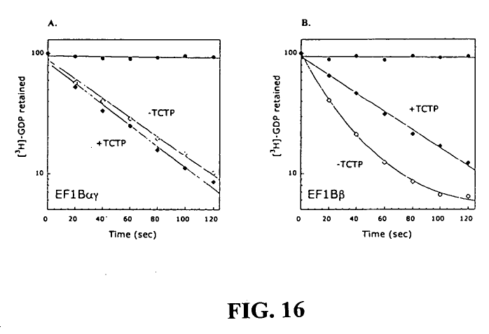

Higher eukaryotes contain two guanine nucleotide exchange factors, eEF1Bα (formerly EF-1β, 27 kDa) and eEF1Bβ (formerly EF-1δ, 35 kDa). The exchange activity of eEF1Bα is enhanced by its association with eEF1Bγ (formerly EF-1γ, 50 kDa). Because TCTP stabilizes the GDP form of eEF1A, its effect on eEF1Bα- or eEF1Bβ-mediated GDP exchange on eEF1A (only eEF1Bβ was found to interact with TCTP in the two-hybrid screen) was investigated. In the presence of the eEF1Bαγ complex GDP exchange followed first-order kinetics (initial rate of 0.84 min⁻¹ per pmol of eEF1Bα≢5; FIG. 16A), whereas eEF1Bβ-mediated exchange was biphasic (initial rates of 0.66 min⁻¹ and 1.68 min⁻¹ per pmol of eEF1Bβ; FIG. 16B). When TCTP was added in the exchange reaction at a final concentration of 2 μM, the kinetics of GDP exchange in the presence of eEF1Bαγ remained essentially unchanged (initial rate of 0.96 min⁻¹; FIG. 16A). By contrast, the initial rate of GDP exchange in the presence of eEF1Bβ and TCTP was no more biphasic and followed a simple first-order rate (0.294 min⁻¹ per pmol of eEF1Bβ; FIG. 16B). These results show that TCTP stabilizes the GDP-form of eEF1A and specifically antagonizes the eEF1Bβ mediated exchange reaction.

6.5 Identification of a Compound that Reduces or Inhibits Binding Between TCTP and Its Binding Partner

Radio-labeled TCTP is generated from an in vitro translation in a rabbit reticulocyte lysate in the presence of ³⁵S-labeled methionine and ³⁵S-labeled cysteine. A GST-fusion protein of a binding partner of TCTP is prepared, and is coupled to sepharose-glutathione beads. ³⁵S labeled TCTP, sepharose-glutathione coupled binding partner and a test compound are mixed and incubated for a sufficient time. A mixture without the test compound is also prepared for control. The binding between TCTP and the binding partner can be quantified using SDS-PAGE or autoradiography.

6.6 Cytotoxic Effects of a Test Compound

One indicator of an anti-cancer agent is its cytotoxic activity. Cytotoxic effects of a test compound can be determined using the following procedures.

Various tumor cell lines (e.g., K562, U937, MDA-DB231, BT20, MCF7) can be used. The cells are seeded at low density and are left for about 24 hours to allow recovery and reach a new logarithmic phase of growth. Various concentrations of the compound being tested are added in triplicate. The mixture is incubated for about 6 days, during which approximately 4 doublings of the cell population occur, to allow the cells to reach subconfluence.

The viability of the cultured cells after the treatment by the test compound is determined by quantifying the level of ATP, since the level of ATP is directly proportional to the number of viable cells present in the culture. The ATP level can be quantified using, for example, CellTiter-Glo® (Promega) luminescent test for cell viability.

6.7 Effects on Cell Apoptosis

The rate of apoptosis can be assessed by using Poly ADP-Ribose Polymerase (PARP) cleavage test. Tumor cells (e.g., U937 cells) are treated with various concentrations of a test compound for 24 hours. The proteins are isolated from these cells and loaded onto a gel for a western blot analysis. A specific anti-PARP antibody is used to visualize the location of PARP. Cleavage of PARP indicates the induction of cell apoptosis.

All of the patents, patent applications and publications referred to in this application are incorporated herein in their entirety by reference. Moreover, citation or identification of any reference in this application is not an admission that such reference is available as prior art to this invention. The full scope of the invention is better understood with reference to the appended claims.

What is claimed is:

1. A method of modulating tumor reversion or cell apoptosis comprising contacting a tumor cell with an effective amount of a proteic binding partner of TCTP.

2. The method of claim 1, wherein the proteic binding partner of TCTP is one of the following:

| PROTEIN | Accession Number |
|---|---|
| Human elongation factor-1 Delta | NM_032378, NM_001960 |
| stathmin | X53305 |
| S100P calcium binding protein | BC006819 |
| casein kinase II alpha subunit | AY112721 |
| beta 2 integrin | BC005861 |
| beta actin | BC016045 |
| prothymosin beta 4 | BC001631 |
| SWI/SNF related, matrix associated, actin | NM_139045 |
| dependent regulator of chromatin | |
| (subfamily a, member 2) | |
| ATP synthase, H+ transporting, | BC022865 |
| mitochondrial F1 complex, O subunit | |
| NADH dehydrogenase (ubiquinone), 1 beta | BC009796 |
| subcomplex 5 | |
| pyruvate dehydrogenase (lipoamide) alpha 1 | BC002406 |
| YME-1 like 1 | BC024032 |
| vascular protein sorting 35 (VPS 35) | NM_018206 |
| ADP-ribosylation factor 1 (ARF 1) | XM_047545 |
| guanine nucleotide binding protein (G | BC021993 |
| protein), beta polypeptide 2-like 1 | |
| coatomer protein complex subunit alpha | U24105 |
| (COPA) | |
| DEAE/H(Asp-Glu-Ala-Asp/His) box | BC016027 |
| polypeptide 5 (RNA helicase) | |
| PAI-1 mRNA binding protein (PAI-RBP1) | NM_015640 |
| Isoleucine t-RNA synthetase | BC008318 |
| cMyb protooncogene | AF104863 |
| Activated RNA polymerase II transcription | BC022339 |
| cofactor 4 | |
| Transcriptional regulating protein 132 | AF297872 |
| (TreP-132) | |
| Eukaryotic translation initiation factor 3, | BC000386 |
| gamma subunit | |
| Topoisomerase II alpha | J04088 |
| Growth factor independence I | BC032751 |
| C-terminal end of the | BC018641 |
| cds-elongation factor I alpha | |
| tRNA nucleotidyl transferase, CCA-adding | AF151805 |
| 1 (TRNT1) | |
| Splicing factor, arginine/serine rich 2 | M90104 |
| Hypoxanthine phosphoribosyltrasferase 1 | BC000578 |
| Dyskerin | BC010015 |
| Human stress-induced phosphoprotein 1 | BC002967 |
| Hsp 70 like protein | AF112210 |
| SH3 domain kinase binding protein 1 | BC015806 |
| (SH3KBP1) | |
| Protein kinase C inhibitor 1 | HSU27143 |
| Pyruvate kinase | BC007952 |
| Glutamate ammonia ligase | BC011852 |
| (glutamine synthase) | |
| Galactosyltransferase I | BC007317 |
| DCAL-1 | NM_172004 |
| Down syndrome critical region 2 | AJ006291 |
| NS-associated protein 1 like protein | AF155568 |
| MHC HLA class I cell surface antigen | M32319 |
| TCTP like protein | BC022436 |
| DHHC1 protein | AF441791 |
| Diazepam binding inhibitor | M14200 |
| HIRA interacting protein 5 | AJ132584 |
| TCAA3 (transforming acidic coiled-coil | AF093543 |
| containing protein 3) | |
| Rad 50 interacting protein | AF317622 |
| Structure specific recognition protein | BC005116 |
| Macrophage galactose-type C-type lectin 2 | XM_090246 |
| like protein | |

3. A method of identifying a compound that alters binding between TCTP and a proteic binding partner of TCTP comprising:
(a) contacting a candidate compound with TCTP and the proteic binding partner of TCTP; and
(b) determining whether the binding between TCTP and the proteic binding partner of TCTP is altered as compared to the binding in the absence of the compound.

4. The method of claim 3, wherein the alteration is decrease or reduction of the binding.

5. The method of claim 3, wherein the alteration is increase or promotion of the binding.

6. The method of claim 3, wherein the proteic binding partner of TCTP is one of the following:

| PROTEIN | Accession Number |
|---|---|
| Human elongation factor-1 Delta | NM_032378, NM_001960 |
| stathmin | X53305 |
| S100P calcium binding protein | BC006819 |
| casein kinase II alpha subunit | AY112721 |
| beta 2 integrin | BC005861 |
| beta actin | BC016045 |
| prothymosin beta 4 | BC001631 |
| SWI/SNF related, matrix associated, actin | NM_139045 |
| dependent regulator of chromatin | |
| (subfamily a, member 2) | |
| ATP synthase, H+ transporting, | BC022865 |
| mitochondrial F1 complex, O subunit | |
| NADH dehydrogenase (ubiquinone), 1 beta | BC009796 |
| subcomplex 5 | |
| pyruvate dehydrogenase (lipoamide) alpha 1 | BC002406 |
| YME-1 like 1 | BC024032 |
| vascular protein sorting 35 (VPS 35) | NM_018206 |
| ADP-ribosylation factor 1 (ARF 1) | XM_047545 |
| guanine nucleotide binding protein (G | BC021993 |
| protein), beta polypeptide 2-like 1 | |
| coatomer protein complex subunit alpha | U24105 |
| (COPA) | |
| DEAE/H(Asp-Glu-Ala-Asp/His) box | BC016027 |
| polypeptide 5 (RNA helicase) | |
| PAI-1 mRNA binding protein (PAI-RBP1) | NM_015640 |
| Isoleucine t-RNA synthetase | BC008318 |
| cMyb protooncogene | AF104863 |
| Activated RNA polymerase II transcription | BC022339 |
| cofactor 4 | |
| Transcriptional regulating protein 132 | AF297872 |
| (TreP-132) | |
| Eukaryotic translation initiation factor 3, | BC000386 |
| gamma subunit | |
| Topoisomerase II alpha | J04088 |
| Growth factor independence I | BC032751 |
| C-terminal end of the | BC018641 |
| cds-elongation factor I alpha | |
| tRNA nucleotidyl transferase, CCA-adding | AF151805 |
| 1 (TRNT1) | |
| Splicing factor, arginine/serine rich 2 | M90104 |
| Hypoxanthine phosphoribosyltrasferase 1 | BC000578 |
| Dyskerin | BC010015 |
| Human stress-induced phosphoprotein 1 | BC002967 |
| Hsp 70 like protein | AF112210 |
| SH3 domain kinase binding protein 1 | BC015806 |
| (SH3KBP1) | |
| Protein kinase C inhibitor 1 | HSU27143 |
| Pyruvate kinase | BC007952 |
| Glutamate ammonia ligase | BC011852 |
| (glutamine synthase) | |
| Galactosyltransferase I | BC007317 |
| DCAL-1 | NM_172004 |
| Down syndrome critical region 2 | AJ006291 |
| NS-associated protein 1 like protein | AF155568 |
| MHC HLA class I cell surface antigen | M32319 |
| TCTP like protein | BC022436 |
| DHHC1 protein | AF441791 |
| Diazepam binding inhibitor | M14200 |
| HIRA interacting protein 5 | AJ132584 |
| TCAA3 (transforming acidic coiled-coil | AF093543 |
| containing protein 3) | |
| Rad 50 interacting protein | AF317622 |
| Structure specific recognition protein | BC005116 |
| Macrophage galactose-type C-type lectin 2 | XM_090246 |
| like protein | |

7. The method of claim 6, wherein the proteic binding partner is stathmin or eEF1A.

8. A method of identifying a compound useful for the treatment, prevention, and management of cancer comprising:
(a) contacting a candidate compound with TCTP and a proteic binding partner of TCTP;
(b) determining whether binding between TCTP and the proteic binding partner of TCTP is altered as compared to the binding in the absence of the compound;
and wherein the binding between TCTP and the proteic binding partner is altered, then
(c) contacting the compound with a tumor cell; and
(d) determining whether tumor reversion or cell apoptosis is increased as compared to a tumor cell that has not been contacted with the compound;
wherein tumor reversion of cell apoptosis is increased, the compound is useful for the treatment, prevention, or management of cancer.

9. The method of claim 8, wherein the proteic binding partner of TCTP is one of the following:

| PROTEIN | Accession Number |
|---|---|
| Human elongation factor-1 Delta | NM_032378, NM_001960 |
| stathmin | X53305 |
| S100P calcium binding protein | BC006819 |
| casein kinase II alpha subunit | AY112721 |
| beta 2 integrin | BC005861 |
| beta actin | BC016045 |
| prothymosin beta 4 | BC001631 |
| SWI/SNF related, matrix associated, actin | NM_139045 |
| dependent regulator of chromatin | |
| (subfamily a, member 2) | |
| ATP synthase, H+ transporting, | BC022865 |
| mitochondrial F1 complex, O subunit | |
| NADH dehydrogenase (ubiquinone), 1 beta | BC009796 |
| subcomplex 5 | |
| pyruvate dehydrogenase (lipoamide) alpha 1 | BC002406 |
| YME-1 like 1 | BC024032 |
| vascular protein sorting 35 (VPS 35) | NM_018206 |
| ADP-ribosylation factor 1 (ARF 1) | XM_047545 |
| guanine nucleotide binding protein (G | BC021993 |
| protein), beta polypeptide 2-like 1 | |
| coatomer protein complex subunit alpha | U24105 |
| (COPA) | |
| DEAE/H(Asp-Glu-Ala-Asp/His) box | BC016027 |
| polypeptide 5 (RNA helicase) | |
| PAI-1 mRNA binding protein (PAI-RBP1) | NM_015640 |
| Isoleucine t-RNA synthetase | BC008318 |
| cMyb protooncogene | AF104863 |
| Activated RNA polymerase II transcription | BC022339 |
| cofactor 4 | |
| Transcriptional regulating protein 132 | AF297872 |
| (TreP-132) | |
| Eukaryotic translation initiation factor 3, | BC000386 |
| gamma subunit | |
| Topoisomerase II alpha | J04088 |
| Growth factor independence I | BC032751 |
| C-terminal end of the | BC018641 |
| cds-elongation factor I alpha | |
| tRNA nucleotidyl transferase, CCA-adding | AF151805 |
| 1 (TRNT1) | |
| Splicing factor, arginine/serine rich 2 | M90104 |
| Hypoxanthine phosphoribosyltrasferase 1 | BC000578 |
| Dyskerin | BC010015 |
| Human stress-induced phosphoprotein 1 | BC002967 |
| Hsp 70 like protein | AF112210 |
| SH3 domain kinase binding protein 1 | BC015806 |
| (SH3KBP1) | |
| Protein kinase C inhibitor 1 | HSU27143 |
| Pyruvate kinase | BC007952 |
| Glutamate ammonia ligase | BC011852 |
| (glutamine synthase) | |
| Galactosyltransferase I | BC007317 |
| DCAL-1 | NM_172004 |
| Down syndrome critical region 2 | AJ006291 |
| NS-associated protein 1 like protein | AF155568 |
| MHC HLA class I cell surface antigen | M32319 |
| TCTP like protein | BC022436 |
| DHHC1 protein | AF441791 |
| Diazepam binding inhibitor | M14200 |
| HIRA interacting protein 5 | AJ132584 |
| TCAA3 (transforming acidic coiled-coil | AF093543 |
| containing protein 3) | |
| Rad 50 interacting protein | AF317622 |
| Structure specific recognition protein | BC005116 |
| Macrophage galactose-type C-type lectin 2 | XM_090246 |
| like protein | |

10. The method of claim 9, wherein the proteic binding partner of TCTP is stathmin or eEF1A.

11. A method of treating, preventing or managing cancer comprising administering to a patient in need of such treatment, prevention or management a therapeutically or prophylactically effective amount of the compound identified using the method of claim 8.

12. A method of identifying a compound useful for the treatment, prevention, or management of a neurodegenerative disorder comprising:
(a) contacting a candidate compound with TCTP and a proteic binding partner of TCTP;
(b) determining whether binding between TCTP and the proteic binding partner of TCTP is altered as compared to the binding in the absence of the compound;
and wherein the binding between TCTP and the proteic binding partner is altered, then
(c) contacting the compound with a cell; and
(d) determining whether cell apoptosis is reduced as compared to a cell that has not been contacted with the compound;
wherein cell apoptosis is reduced, the compound is useful for the treatment, prevention, or management of a neurodegenerative disorder.

13. The method of claim 12, wherein the proteic binding partner of TCTP is one of the following:

| PROTEIN | Accession Number |
|---|---|
| Human elongation factor-1 Delta | NM_032378, NM_001960 |
| stathmin | X53305 |
| S100P calcium binding protein | BC006819 |
| casein kinase II alpha subunit | AY112721 |
| beta 2 integrin | BC005861 |
| beta actin | BC016045 |
| prothymosin beta 4 | BC001631 |
| SWI/SNF related, matrix associated, actin | NM_139045 |
| dependent regulator of chromatin | |
| (subfamily a, member 2) | |
| ATP synthase, H+ transporting, | BC022865 |
| mitochondrial F1 complex, O subunit | |
| NADH dehydrogenase (ubiquinone), 1 beta | BC009796 |
| subcomplex 5 | |
| pyruvate dehydrogenase (lipoamide) alpha 1 | BC002406 |
| YME-1 like 1 | BC024032 |
| vascular protein sorting 35 (VPS 35) | NM_018206 |
| ADP-ribosylation factor 1 (ARF 1) | XM_047545 |
| guanine nucleotide binding protein (G | BC021993 |
| protein), beta polypeptide 2-like 1 | |
| coatomer protein complex subunit alpha | U24105 |
| (COPA) | |
| DEAE/H(Asp-Glu-Ala-Asp/His) box | BC016027 |
| polypeptide 5 (RNA helicase) | |
| PAI-1 mRNA binding protein (PAI-RBP1) | NM_015640 |
| Isoleucine t-RNA synthetase | BC008318 |
| cMyb protooncogene | AF104863 |
| Activated RNA polymerase II transcription | BC022339 |
| cofactor 4 | |
| Transcriptional regulating protein 132 | AF297872 |
| (TreP-132) | |
| Eukaryotic translation initiation factor 3, | BC000386 |
| gamma subunit | |
| Topoisomerase II alpha | J04088 |
| Growth factor independence I | BC032751 |
| C-terminal end of the | BC018641 |
| cds-elongation factor I alpha | |
| tRNA nucleotidyl transferase, CCA-adding | AF151805 |
| 1 (TRNT1) | |
| Splicing factor, arginine/serine rich 2 | M90104 |
| Hypoxanthine phosphoribosyltrasferase 1 | BC000578 |
| Dyskerin | BC010015 |
| Human stress-induced phosphoprotein 1 | BC002967 |
| Hsp 70 like protein | AF112210 |
| SH3 domain kinase binding protein 1 | BC015806 |
| (SH3KBP1) | |
| Protein kinase C inhibitor 1 | HSU27143 |
| Pyruvate kinase | BC007952 |
| Glutamate ammonia ligase | BC011852 |
| (glutamine synthase) | |
| Galactosyltransferase I | BC007317 |
| DCAL-1 | NM_172004 |
| Down syndrome critical region 2 | AJ006291 |
| NS-associated protein 1 like protein | AF155568 |
| MHC HLA class I cell surface antigen | M32319 |
| TCTP like protein | BC022436 |
| DHHC1 protein | AF441791 |
| Diazepam binding inhibitor | M14200 |
| HIRA interacting protein 5 | AJ132584 |
| TCAA3 (transforming acidic coiled-coil | AF093543 |
| containing protein 3) | |
| Rad 50 interacting protein | AF317622 |
| Structure specific recognition protein | BC005116 |
| Macrophage galactose-type C-type lectin 2 | XM_090246 |
| like protein | |

14. The method of claim 13, wherein the proteic binding partner of TCTP is stathmin or eEF1A.

15. A method of treating, preventing or managing a neurodegenerative disorder comprising administering to a patient in need of such treatment, prevention or management a therapeutically or prophylactically effective amount of the compound identified using the method of claim 12.

16. The method of claim 15, wherein the neurodegenerative disorder is Alzheimer's disease.

17. A method of identifying a compound useful for the treatment, prevention, or management of an inflammatory disorder comprising:
(a) contacting a candidate compound with TCTP and a proteic binding partner of TCTP;
(b) determining whether binding between TCTP and the proteic binding partner of TCTP is altered as compared to the binding in the absence of the compound;
and wherein the binding between TCTP and the proteic binding partner is altered, then
(c) determining whether the compound has an antiinflammatory activity;
wherein the compound has an antiinflammatory activity, the compound is useful for the treatment, prevention, or management of an inflammatory disorder.

18. The method of claim 17, wherein the proteic binding partner of TCTP is one of the following:

| PROTEIN | Accession Number |
|---|---|
| Human elongation factor-1 Delta | NM_032378, NM_001960 |
| stathmin | X53305 |
| S100P calcium binding protein | BC006819 |
| casein kinase II alpha subunit | AY112721 |
| beta 2 integrin | BC005861 |
| beta actin | BC016045 |
| prothymosin beta 4 | BC001631 |
| SWI/SNF related, matrix associated, actin | NM_139045 |
| dependent regulator of chromatin | |
| (subfamily a, member 2) | |
| ATP synthase, H+ transporting, | BC022865 |
| mitochondrial F1 complex, O subunit | |
| NADH dehydrogenase (ubiquinone), 1 beta | BC009796 |
| subcomplex 5 | |
| pyruvate dehydrogenase (lipoamide) alpha 1 | BC002406 |
| YME-1 like 1 | BC024032 |
| vascular protein sorting 35 (VPS 35) | NM_018206 |
| ADP-ribosylation factor 1 (ARF 1) | XM_047545 |
| guanine nucleotide binding protein (G | BC021993 |
| protein), beta polypeptide 2-like 1 | |
| coatomer protein complex subunit alpha | U24105 |
| (COPA) | |
| DEAE/H(Asp-Glu-Ala-Asp/His) box | BC016027 |
| polypeptide 5 (RNA helicase) | |
| PAI-1 mRNA binding protein (PAI-RBP1) | NM_015640 |
| Isoleucine t-RNA synthetase | BC008318 |
| cMyb protooncogene | AF104863 |
| Activated RNA polymerase II transcription | BC022339 |
| cofactor 4 | |
| Transcriptional regulating protein 132 | AF297872 |
| (TreP-132) | |
| Eukaryotic translation initiation factor 3, | BC000386 |
| gamma subunit | |
| Topoisomerase II alpha | J04088 |
| Growth factor independence I | BC032751 |
| C-terminal end of the | BC018641 |
| cds-elongation factor I alpha | |
| tRNA nucleotidyl transferase, CCA-adding | AF151805 |
| 1 (TRNT1) | |
| Splicing factor, arginine/serine rich 2 | M90104 |
| Hypoxanthine phosphoribosyltrasferase 1 | BC000578 |
| Dyskerin | BC010015 |
| Human stress-induced phosphoprotein 1 | BC002967 |
| Hsp 70 like protein | AF112210 |
| SH3 domain kinase binding protein 1 | BC015806 |
| (SH3KBP1) | |
| Protein kinase C inhibitor 1 | HSU27143 |
| Pyruvate kinase | BC007952 |
| Glutamate ammonia ligase | BC011852 |
| (glutamine synthase) | |
| Galactosyltransferase I | BC007317 |
| DCAL-1 | NM_172004 |
| Down syndrome critical region 2 | AJ006291 |
| NS-associated protein 1 like protein | AF155568 |
| MHC HLA class I cell surface antigen | M32319 |
| TCTP like protein | BC022436 |
| DHHC1 protein | AF441791 |
| Diazepam binding inhibitor | M14200 |
| HIRA interacting protein 5 | AJ132584 |
| TCAA3 (transforming acidic coiled-coil | AF093543 |
| containing protein 3) | |
| Rad 50 interacting protein | AF317622 |
| Structure specific recognition protein | BC005116 |
| Macrophage galactose-type C-type lectin 2 | XM_090246 |
| like protein | |

19. The method of claim 18, wherein the proteic binding partner of TCTP is stathmin or eEF1A.

20. A method of treating, preventing or managing an inflammatory disorder comprising administering to a patient in need of such treatment, prevention or management a therapeutically or prophylactically effective amount of the compound identified using the method of claim 17.

21. A method of determining the cause of cancer, an inflammatory disorder, or a neurodegenerative disorder in a patient suffering from said disorders comprising:
(a) obtaining a first cell from the patient;
(b) determining the binding between TCTP and a proteic binding partner in the first cell;
(c) obtaining a second cell from a normal subject;
(d) determining the binding between TCTP and the proteic binding partner in the second cell; and
(e) comparing the bindings obtained from the first cell and the second cell and determining whether the binding in the first cell is altered;
wherein the binding is altered, the cause of said disorders is attributed to said alteration of binding.

22. A method of diagnosing cancer or predicting the propensity for occurrence of cancer in a subject comprising:
(a) obtaining a first cell from a potential patient;
(b) determining the binding between TCTP and a proteic binding partner in the first cell;
(c) obtaining a second cell from a normal subject;
(d) determining the binding between TCTP and the proteic binding partner in the second cell; and
(e) comparing the bindings obtained from the first cell and the second cell and determining whether the binding in the first cell is altered;
wherein the binding is altered, the potential patient is diagnosed for cancer or has a higher than normal propensity for the occurrence of cancer.

23. The method of claim 21 or 22, wherein the proteic binding partner of TCTP is one of the following:

| PROTEIN | Accession Number |
|---|---|
| Human elongation factor-1 Delta | NM_032378, NM_001960 |
| stathmin | X53305 |
| S100P calcium binding protein | BC006819 |
| casein kinase II alpha subunit | AY112721 |
| beta 2 integrin | BC005861 |
| beta actin | BC016045 |
| prothymosin beta 4 | BC001631 |
| SWI/SNF related, matrix associated, actin | NM_139045 |
| dependent regulator of chromatin | |
| (subfamily a, member 2) | |
| ATP synthase, H+ transporting, | BC022865 |
| mitochondrial F1 complex, O subunit | |
| NADH dehydrogenase (ubiquinone), 1 beta | BC009796 |
| subcomplex 5 | |
| pyruvate dehydrogenase (lipoamide) alpha 1 | BC002406 |
| YME-1 like 1 | BC024032 |
| vascular protein sorting 35 (VPS 35) | NM_018206 |
| ADP-ribosylation factor 1 (ARF 1) | XM_047545 |
| guanine nucleotide binding protein (G | BC021993 |
| protein), beta polypeptide 2-like 1 | |
| coatomer protein complex subunit alpha | U24105 |
| (COPA) | |
| DEAE/H(Asp-Glu-Ala-Asp/His) box | BC016027 |
| polypeptide (RNA helicase) | |
| PAI-1 mRNA binding protein (PAI-RBP1) | NM_015640 |
| Isoleucine t-RNA synthetase | BC008318 |
| cMyb protooncogene | AF104863 |
| Activated RNA polymerase II transcription | BC022339 |
| cofactor 4 | |
| Transcriptional regulating protein 132 | AF297872 |
| (TreP-132) | |
| Eukaryotic translation initiation factor 3, | BC000386 |
| gamma subunit | |
| Topoisomerase II alpha | J04088 |
| Growth factor independence I | BC032751 |
| C-terminal end of the | BC018641 |
| cds-elongation factor I alpha | |
| tRNA nucleotidyl transferase, CCA-adding | AF151805 |
| 1 (TRNT1) | |
| Splicing factor, arginine/serine rich 2 | M90104 |
| Hypoxanthine phosphoribosyltrasferase 1 | BC000578 |
| Dyskerin | BC010015 |
| Human stress-induced phosphoprotein 1 | BC002967 |
| Hsp 70 like protein | AF112210 |
| SH3 domain kinase binding protein 1 | BC015806 |
| (SH3KBP1) | |
| Protein kinase C inhibitor 1 | HSU27143 |
| Pyruvate kinase | BC007952 |
| Glutamate ammonia ligase | BC011852 |
| (glutamine synthase) | |
| Galactosyltransferase I | BC007317 |
| DCAL-1 | NM_172004 |
| Down syndrome critical region 2 | AJ006291 |
| NS-associated protein 1 like protein | AF155568 |
| MHC HLA class I cell surface antigen | M32319 |
| TCTP like protein | BC022436 |
| DHHC1 protein | AF441791 |
| Diazepam binding inhibitor | M14200 |
| HIRA interacting protein 5 | AJ132584 |
| TCAA3 (transforming acidic coiled-coil | AF093543 |
| containing protein 3) | |
| Rad 50 interacting protein | AF317622 |
| Structure specific recognition protein | BC005116 |
| Macrophage galactose-type C-type lectin 2 | XM_090246 |
| like protein | |

24. The method of claim 23, wherein the proteic binding partner of TCTP is stathmin or OP18.


SEQUENCE LISTING (39 sequences)

SEQ ID NO: 1 - DNA, 21 bp, Unknown
  siRNA TCTP (chemically synthesized)
aagguaccgaaagcacaguaa

SEQ ID NO: 2 - DNA, 21 bp, Unknown
  siRNA GFP (chemically synthesized)
aacacuggucacuaccuucac

SEQ ID NO: 3 - protein, 8 aa, Artificial Sequence
  Synthetic peptide
QLIVGVNK

SEQ ID NO: 4 - protein, 7 aa, Artificial Sequence
  Synthetic peptide
FAVRDMR

SEQ ID NO: 5 - protein, 9 aa, Artificial Sequence
  Synthetic peptide
QTVAVGVIK

SEQ ID NO: 6 - protein, 8 aa, Artificial Sequence
  Synthetic peptide
LPLQDVYK

SEQ ID NO: 7 - protein, 11 aa, Artificial Sequence
  Synthetic peptide
IGGIGTVPVGR

SEQ ID NO: 8 - protein, 12 aa, Artificial Sequence
  Synthetic peptide
EHALLAYTLGVK

SEQ ID NO: 9 - protein, 11 aa, Artificial Sequence
  Synthetic peptide
YYVIIDAPGHR

SEQ ID NO: 10 - protein, 24 aa, Artificial Sequence
  Synthetic peptide
VETGVLKPGMVVTFAPVNVTTEVK

SEQ ID NO: 11 - protein, 24 aa, Artificial Sequence
  Synthetic peptide
VETGVLKPGMVVTFAPVNVTTEVK

SEQ ID NO: 12 - protein, 28 aa, Artificial Sequence
  Synthetic peptide
NMITGTSQADCAVLIVAAGVEFEAGISK

SEQ ID NO: 13 - protein, 28 aa, Artificial Sequence
  Synthetic peptide
SGDAAIVDMVPGKPMCVESFSDYPPLGR

SEQ ID NO: 14 - protein, 28 aa, Artificial Sequence
  Synthetic peptide
DGNASGTTLLEALDCILPPTRPTDKPLR

SEQ ID NO: 15 - protein, 8 aa, Artificial Sequence
  Synthetic peptide
FSVSPVVR

SEQ ID NO: 16 - protein, 8 aa, Artificial Sequence
  Synthetic peptide
SDPVVSYR

SEQ ID NO: 17 - protein, 10 aa, Artificial Sequence
  Synthetic peptide
GGGQIIPTAR

SEQ ID NO: 18 - protein, 10 aa, Artificial Sequence
  Synthetic peptide
GEGQLGPAER

SEQ ID NO: 19 - protein, 9 aa, Artificial Sequence
  Synthetic peptide
GPLMMYISK

SEQ ID NO: 20 - protein, 9 aa, Artificial Sequence
  Synthetic peptide
VFDAIMNFK

SEQ ID NO: 21 - protein, 9 aa, Artificial Sequence
  Synthetic peptide
YEMDVAEAR

SEQ ID NO: 22 - protein, 10 aa, Artificial Sequence
  Synthetic peptide
EGALCEENMR

SEQ ID NO: 23 - protein, 10 aa, Artificial Sequence
  Synthetic peptide
EDLYLKPIQR

SEQ ID NO: 24 - protein, 12 aa, Artificial Sequence
  Synthetic peptide
NMSVIAHVDHGK

SEQ ID NO: 25 - protein, 12 aa, Artificial Sequence
  Synthetic peptide
CLYASVLTAQPR

SEQ ID NO: 26 - protein, 11 aa, Artificial Sequence
  Synthetic peptide
KEDLYLKPIQR

SEQ ID NO: 27 - protein, 12 aa, Artificial Sequence
  Synthetic peptide
IKPVLMMNKMDR

SEQ ID NO: 28 - protein, 12 aa, Artificial Sequence
  Synthetic peptide
IKPVLMMNKMDR

SEQ ID NO: 29 - protein, 14 aa, Artificial Sequence
  Synthetic peptide
TGTTTTTEHAHNMR

SEQ ID NO: 30 - protein, 13 aa, Artificial Sequence
  Synthetic peptide
TGTTTTTEHANMR

SEQ ID NO: 31 - protein, 16 aa, Artificial Sequence
  Synthetic peptide
AYLPVNESFGFTADLR

SEQ ID NO: 32 - protein, 18 aa, Artificial Sequence
  Synthetic peptide
GHVFEESQVAGTPMFVVK

SEQ ID NO: 33 - protein, 18 aa, Artificial Sequence
  Synthetic peptide
GHVFEESQVAGTPMFVVK

SEQ ID NO: 34 - protein, 19 aa, Artificial Sequence
  Synthetic peptide
RGHVFEESQVAGTPMFVVK

SEQ ID NO: 35 - protein, 20 aa, Artificial Sequence
  Synthetic peptide
ARPFPDGLAEDIDKGEVSAR

SEQ ID NO: 36 - protein, 19 aa, Artificial Sequence
  Synthetic peptide
STAISLFYELSENDLNFTK

SEQ ID NO: 37 - protein, 18 aa, Artificial Sequence
  Synthetic peptide
ALLELQLEPEELYQTFQR

SEQ ID NO: 38 - protein, 21 aa, Artificial Sequence
  Synthetic peptide
GVQYLNEIKDSVVAGFQWATK

SEQ ID NO: 39 - protein, 27 aa, Artificial Sequence
  Synthetic peptide
DGAGFLINLIDSPGHVDFSSEVTAALR